(19) United States
(12) Patent Application Publication
(10) Pub. No.: US 2013/0338018 A1
(43) Pub. Date: Dec. 19, 2013

(54) PRIMERS, SEQUENCES AND RECOMBINANT PROBES FOR IDENTIFICATION OF MYCOBACTERIUM SPECIES

(71) Applicants: Jianli Dai, Columbia, MD (US); J. Glenn Morris, Micanopy, FL (US)

(72) Inventors: Jianli Dai, Columbia, MD (US); J. Glenn Morris, Micanopy, FL (US)

(73) Assignee: UNIVERSITY OF FLORIDA RESEARCH FOUNDATION INC., Gainesville, FL (US)

(21) Appl. No.: 13/521,504

(22) PCT Filed: Jan. 13, 2011
(86) PCT No.: PCT/US11/21087

§ 371 Date: Jul. 11, 2012

Related U.S. Application Data
(60) Provisional application No. 61/297,924, filed on Jan. 25, 2010.

(51) Int. Cl. C12Q 1/68

(52) U.S. Cl. CPC C12Q 1/689
USPC 506/9; 435/6.11; 435/6.15; 435/6.12; 536/24.32; 536/24.33; 506/16

(57) ABSTRACT

The subject invention pertains to an assay and a method for diagnosing, identifying and/or differentiating microorganisms, and in particular bacteria such as Mycobacterium spp. within biological samples. The present invention also relates to assays, gene arrays, probes and primers, nucleic acids and methods for detecting microorganisms in a sample.

### CROSS-REFERENCE TO RELATED APPLICATION

The present application claims the benefit of U.S. Provisional Application Ser. No. 61/297,924, filed Jan. 25, 2010, which is hereby incorporated by reference herein in its entirety, including any figures, tables, or drawings.

### BACKGROUND OF THE INVENTION

Mycobacteria, Gram-positive, aerobic bacteria characterized by a thick hydrophobic, waxy cell wall, are important causes of morbidity and mortality worldwide. Mycobacterium tuberculosis (MTB) and M. leprae are the best known and most virulent species. They were discovered in late 19th century (54). The infections by nontuberculous mycobacteria (NTM) have only been recognized for about 70 years and are a growing cause for concerns (68). NTM are ubiquitous environmental organisms, some of which cause severe respiratory diseases as well as other infection in human, especially those with immunodeficiency. In contrast to MTB, the incidence of NTM infections in the U.S. has risen steadily over the last several decades and has now surpassed that of MTB (2, 4, 8, 29). The number of NTM species identified has been increasing dramatically from 39 in 1996 (8) to the current 142 (www.bacterio.cict.fr/m/mycobacterium.html).

Culture based identification methods using biochemical tests are slow and inadequate to differentiate this growing list of species. Molecular methods are beginning to be developed, but many loci used are not present in all NTM. NTM are also often resistant to multiple antimicrobial agents. To improve our ability to diagnose and treat NTM infections, we need better molecular diagnostic tests. Accurate identification of organisms will increase our understanding of the resistance and virulence of individual Mycobacterium spp. In addition, molecular typing tools are needed for epidemiologic studies. All of these are limited by our lack of understanding of the population structure and genetic variability of NTM.

Several loci have been used to type mycobacteria including 16S rDNA (7, 24, 32, 51), 16S-23S rDNA internal transcribed spacer (ITS) (13, 18, 53, 66), hsp65 (31, 63), gyrB (21, 28, 46), rpoB (20, 30, 36), dnaJ1 (60, 70), recA (3, 67), sodA (76), secA1 (74), tuf (40), ssrA (40), smpB (41) and a 32-kDa protein gene (55, 56). However, these loci are either not detected in all species necessitating sequencing of multiple loci for identification of isolates or they are not specifically discriminatory to differentiate closely related species. For example, the widely used 16S rDNA typing can not differentiate the pathogen M. kansasii from the non-pathogen M. gastri (49, 65), M. marinum from M. ulcerans, M. fortuitum from M. acetamidolyticum, and species within the Mycobacterium tuberculosis Complex (MTC) and Mycobacterium avium Complex (65). M. marinum and M. ulcerans even have identical ITS sequence (53).

To further complicate matters, some mycobacterial species have two different rRNA operons, resulting in ambiguous 16s rDNA (47, 50, 65) and ITS sequences (57). The gyrB locus was tested only in slow growing mycobacteria (SGM) (21, 28, 46) and needs further study on rapidly growing mycobacteria (RGM). Primers for dnaJ1 have difficulty amplifying DNA from MTB and M. intermedium (70). Similar primer failure has been reported for the sodA locus with at least 15 species (40). The hsp65 sequences are more conserved than other loci, except 16s rDNA (51), making it an easy target to amply, but it is unable to differentiate the members of MTC as well as M. simiae from M. genavense (31). Multilocus sequence is an approach to overcome the shortcomings the single locus methods mentioned above and proved to be a very useful tool to identify species as well as study evolution of the Mycobacterium genus (14, 41), but more loci are needed. In this study, we used multiple genome comparison to systemically locate potential typing loci for Mycobacterium.

### BRIEF SUMMARY OF THE INVENTION

The present invention relates to an assay and a method for diagnosing, identifying and/or differentiating microorganisms, and in particular bacteria such as Mycobacterium spp. within biological samples. The present invention also relates to assays, gene arrays, probes and primers, nucleic acids and methods for detecting microorganisms in a sample.

### BRIEF DESCRIPTION OF THE DRAWINGS

FIGS. 1A-1B. FIG. 1A. Neighbor-Joining tree of 26 CHRs from 18 mycobacterial genomes rooted with N. farcinica IFM 10152. Two M. bovis and four M. tuberculosis are compressed into MTC. The percentages of replicate trees in a bootstrap test of 2000 replicates are shown at the branches. Complete deletion option for gaps is used. FIG. 1B. The expanded subtree of MTC. SGM: Slowly growing mycobacteria. RGM: Rapidly growing mycobacteria.

FIGS. 2A-2C. Single gene Neighbor-Joining phylogenic trees rooted with N. farcinica IFM 10152 (FIG. 2A. rpoBC; FIG. 2B. dnaK; FIG. 2C. hsp65). The percentages of bootstrap values are shown next to the nodes. SGM misplaced into RGM are marked with “*” at the end of their names.

FIG. 3. Neighbor-Joining phylogenic tree of concatenated dnaK, hsp65, and rpoBC loci. The tree is rooted with N. farcinica IFM 10152. SGM misplaced into RGM clade is marked with “*”.

FIG. 4. Neighbor-joining unrooted tree of 16S rDNA from species related to M. sp. USFLJA0011. Complete deletion option was used for gaps in the alignment. Bootstrap values are shown at the node. The typed strains are ended with “T”.

FIG. 5. Clustal alignment of the rpoBC region of the 27 sequenced genomes in the suborder Corynebacterineae. The aligned sequences cover the last rpoB CHRs, the first rpoC CHRs, and the sequences between them. Species names are truncated to 30 characters. Bases identical to M. tuberculosis H37Rv are shown as “.” and gaps are shown as “-”. The positions with identical bases in all sequences are marked with “*” in Clustal Consensus. The two underlined regions are targets of amplification/sequencing primers. The stop codons of rpoB and the start codon of rpoC are highlighted.

FIG. 6. Clustal alignment of the dnaK region of the 27 sequenced genomes of the suborder Corynebacterineae. The aligned sequences cover the two adjacent dnaK CHRs and the sequences between them. Species names are truncated to 30 characters. Bases identical to M. tuberculosis H37Rv are shown as “.” and gaps are shown as “-”. The positions with identical bases in all sequences are marked with “*” in Clustal Consensus. There are two dnaK paralogs in R. Jostii RHA1, dnaK1 and dnaK4. Both of them are included in the alignment. The two underlined regions are targets of amplification/sequencing primers.

### DETAILED DISCLOSURE OF THE INVENTION

The following definitions serve to illustrate the terms and expressions used in the different embodiments of the present invention as set out below.

An isolated nucleic acid molecule is one which is separated from other nucleic acid molecules which are present in the natural source of the nucleic acid. For example, with regards to genomic DNA, the term isolated includes nucleic acid molecules which are separated from the chromosome with which the genomic DNA is naturally associated.

The term probe or nucleic acid probe refers to single stranded sequence-specific oligonucleotides which have a base sequence which is sufficiently complementary to hybridize to the target base sequence to be detected (in this case, any one of SEQ ID NOs: 1-46).

The term primer refers to a single stranded DNA oligonucleotide sequence capable of acting as a point of initiation for synthesis of a primer, extension product which is complementary to the nucleic acid strand to be copied. The length and the sequence of the primer must be such that they allow to prime the synthesis of the extension products. In certain embodiments, primers are about 5-50 nucleotides long. Specific length and sequence will depend on the complexity of the required DNA or RNA targets, as well as on the conditions of primer use such as temperature and ionic strength.

The term “target” or “target sequence” refers to nucleic acid molecules originating from a biological sample which have a base sequence complementary to the nucleic acid probe of the invention. The target nucleic acid can be single-or double-stranded DNA (if appropriate, obtained following amplification) and contains a sequence which has at least partial complementarily with at least one probe oligonucleotide.

The phrase a (biological) sample refers to a specimen such as a clinical sample from a human or animal, an environmental sample, bacterial colonies, contaminated or pure cultures or purified nucleic acid in which the target sequence of interest may be found.

The present invention relates to an assay for detecting and identifying one or more microorganisms in a sample, characterized in that said assay comprises the use of at least two genetic regions/loci. Preferably said micro-organisms are bacterial species of the genera Mycobacterium, Corynebacterium, Nocardia and/or Rhodococcus. In a preferred embodiment, the assay of the present invention is characterized in that it comprises the use of at least one genetic region/locus.

In accordance with the present invention a number of genetic regions/loci were identified and characterized which are extremely suitable for permitting the detection and identification genotyping bacterial species in the genera Mycobacterium, Corynebacterium, Nocardia and/or Rhodococcus.

In one aspect of the invention, the assays and arrays described herein utilize one or more of the loci disclosed in Table 3. Thus, assays and arrays of the present invention comprise polynucleotides that hybridize with the loci disclosed in Table 3. In certain embodiments of the invention, the assays and arrays utilize polynucleotides that hybridize with fragments of that contain the regions between the CHRs identified in Table 3.

In another aspect of the invention, the assays and arrays described herein utilize one or more of the loci disclosed herein. In a one embodiment, the assays and arrays of the present invention comprise polynucleotides that hybridize with a polynucleotide comprising SEQ ID NO: 3 (dnaK locus) and/or a rpoBC locus that comprises SEQ ID NO: 1. In certain embodiments of the invention, the assays and arrays utilize fragments of SEQ ID NOs: 1 and 3 that contain the regions between the end or one CHR and the start of another (as identified in Table 3). Examples of such regions are also found in SEQ ID NOs: 1 and 3. As noted in Table 3, the numbering of the start and end positions are based upon the M. tuberculosis H37Rv genome disclosed in GenBank Accession No. NC_{—}000962, which is hereby incorporated by reference in its entirety.

Yet another aspect of the invention provides an array of polynucleotides that comprises one or more of the following polynucleotide sequences: SEQ ID NO: 19, 20, 21, 22, 23, 24, 25, 26, 27, 28, 29, 30, 31, 32, 33, 34, 35, 36, 37, 38, 39, 40, 41, 42, 43, 44, 45 or 46. In certain aspects of the invention, the array comprises any 2, 3, 4, 5, 6, 7, 8, 9, 10, 11, 12, 13, 14, 15, 16, 17, 18, 19, 20, 21, 22, 23, 24, 25, 26 or 27 (or all) of the aforementioned sequences.

In a further aspect, the present invention provides conserved nucleic acid sequences for the detection and/or identification of one or more microorganisms. These nucleic acid sequences are selected from any one of SEQ ID NOs: 1-46. Various embodiments also provide for linking various sequences into a single sequence (joined by a nucleotide linker sequences. For example, SEQ ID NO: 18 may be joined to SEQ ID NO: 19 by a nucleotide linker sequence.

Primers and probes can also be derived from SEQ ID NOs: 1-46. Thus, the invention provides primer pairs (forward and reverse primers) suitable for amplifying a locus (e.g., any one of SEQ ID NO: 1-46 or 18-46). The primers of the present invention are at least 9 nucleotides in length and can be as long as about 50 nucleotides. In various embodiments, the primer may be, for example, least 15 nucleotides in length and has at least 70%, 80%, 90% or more than 95% identity to the full complement of the target sequence. Of course, primers consisting of more than 50 nucleotides can be used.

The present invention also relates to a nucleic acid probe capable of hybridizing to a locus described herein (e.g., any one of SEQ ID NO: 1-46 or 18-46). As described herein, probes are at least 9 nucleotides in length and have at least 70%, 80%, 90% or more than 95% identity to the complement of the target sequence to be detected. In certain preferred embodiments, probes are about 15 to 50 nucleotides long. As also disclosed herein, the primers and probes can be used for diagnostic purposes, in investigating the presence or the absence of a target nucleic acid in a biological sample, according to all the known hybridization techniques such as for instance dot blot, slot blot, hybridization on arrays, etc. The probes of the invention will preferably hybridize specifically to one or more of the above-mentioned loci.

The nucleic acid probes of this invention can be included in a composition or kit which can be used to rapidly determine the presence or absence of pathogenic species of interest (see below).

Yet another aspect of the invention relates to an assay for detecting and identifying one or more microorganisms in a sample, characterized in that said assay comprises the use of at least one of the genetic regions/loci disclosed herein. Preferably the microorganisms are bacterial species of the genera Mycobacterium, Corynebacterium, Nocardia and/or Rhodococcus. In accordance with the present invention a number of genetic regions/loci were identified and characterized which are extremely suitable for permitting the detection and identification genotyping bacterial species in the genera Mycobacterium, Corynebacterium, Nocardia and/or Rhodococcus (see, for example, Table 3).

Thus, one aspect of the invention provides the assays and arrays that utilize one or more of the loci disclosed in Table 3. Thus, assays and arrays of the present invention comprise polynucleotides that hybridize with the loci disclosed in Table 3.

In another aspect of the invention, the assays and arrays described herein utilize one or more of the loci disclosed herein. In a one embodiment, the assays and arrays of the present invention comprise polynucleotides that hybridize with a polynucleotide comprising SEQ ID NO: 3 (dnaK locus) and/or a rpoBC locus that comprises SEQ ID NO: 1. As noted in Table 3, the numbering of the start and end positions of genetic loci disclosed therein are based upon the M. tuberculosis H37Rv genome disclosed in GenBank Accession No. NC_{—}000962, which is hereby incorporated by reference in its entirety.

Yet another aspect of the invention provides an array of polynucleotides that comprises one or more of the following polynucleotide sequences: SEQ ID NO: 19, 20, 21, 22, 23, 24, 25, 26, 27, 28, 29, 30, 31, 32, 33, 34, 35, 36, 37, 38, 39, 40, 41, 42, 43, 44, 45 or 46. In certain aspects of the invention, the array comprises any 2, 3, 4, 5, 6, 7, 8, 9, 10, 11, 12, 13, 14, 15, 16, 17, 18, 19, 20, 21, 22, 23, 24, 25, 26 or 27 (or all) of the aforementioned sequences affixed to a solid support.

### Compositions and Kits

In another aspect of the invention, compositions and kits comprising the disclosed loci, primers and/or probes are provided. Thus, a composition comprising at least one primer pair (forward and reverse primers) suitable for amplifying a locus is provided. In yet another aspect of the invention, embodiment, the invention relates to a composition comprising at least one nucleic acid probe capable of hybridizing to a locus disclosed herein. By composition, it is meant that primers or probes complementary to the loci described herein may be in a pure state or in combination with other primers or probes. In addition, the primers or probes may be in combination with salts or buffers, and may be in a dried state, in an alcohol solution as a precipitate, or in an aqueous solution.

In yet another embodiment, the invention relates to a kit for detecting and identifying one or more microorganisms in a sample. Thus, kits may comprise: a) a composition comprising at least one primer pair (forward and reverse primers) suitable for amplifying a locus described herein; b) a composition comprising at least one nucleic acid probe capable of hybridizing to a locus described herein; c) a buffer suitable for hybridization reactions between the probes or primers and nucleic acid targets in a sample; d) a solution for washing hybridized nucleic acids formed under the appropriate wash conditions or components necessary for producing the solution, and e) optionally a means for detection of said hybrids.

### Arrays

In another embodiment, the present invention provides an array of nucleic acids immobilized on a solid support. Thus, one embodiment provides for an array of nucleic acids comprising any one or more of SEQ ID NOs: 1-17 immobilized on a solid support. Another embodiment provides for an array of nucleic acids comprising any one or more of SEQ ID NOs: 18-46 immobilized on a solid support.

In another embodiment, the present invention provides an array of probes and/or primers immobilized on a solid support. Thus, one embodiment provides for an array of probes and/or primers immobilized on a solid support such that the probe and/or primer hybridizes with any one or more of SEQ ID NOs: 1-46. Another embodiment provides for an array of nucleic acids comprising any one or more of SEQ ID NOs: 18-46 immobilized on a solid support.

Examples of a solid support on which the array or nucleic acids may be immobilized include, and are not limited to, materials such as paper, glass, silicon and polymeric materials such as acryl, polyethylene terephtalate (PET), polystyrene, polycarbonate and polypropylene. The nucleic acids may be immobilized on the substrate by a covalent bond at either 3′ end or 5′ end. The immobilization can be achieved by conventional techniques, for example, using electrostatic force, binding between aldehyde coated slide and amine group attached on synthetic oligomeric phase or spotting on amine coated slide, L-lysine coated slide or nitrocellulose coated slide. The immobilization and the arrangement of nucleic acids onto a solid substrate may be carried out by pin microarray, inkjet, photolithography, electric array, etc. The term DNA chip as used herein, is to be understood in its broadest sense, i.e. including nanochips or nanotools that are designed to recognize a specific pattern of nucleic acids through hybridization.

### Assays

In another embodiment, the invention relates to an assay for detecting and identifying one or more microorganisms in a sample. In various embodiments, the assay comprises the use of one or more or the disclosed loci to distinguish detect and identify a microorganism.

The disclosed assays provide a means by which the genus, specie, and optionally strain, of a microorganism within a sample may be identified. In certain embodiments, the assays comprise the amplification of genetic loci and the hybridization of amplicons to specific probes covalently bound on an array or, alternatively, to hybridize a probe during the amplification step (e.g. real time PCR with Taqman or molecular Beacon probes). Thus, in one embodiment, the method for detecting and identifying one or more microorganism comprise the following steps:

a) optionally isolating and/or concentrating the DNA present in a sample;

b) amplifying said DNA with at least one pair of (forward and reverse) primers suitable for amplifying a locus described herein;

c) hybridizing the amplified DNA fragments obtained in step b) with a probe or primer that hybridizes with a locus as described herein;

d) detecting the hybrids formed in step c); and

e) identifying microorganisms in said sample from the hybridization signals obtained in step d).

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### Genome Comparison for Mycobacterial Typing Loci

### Materials and Methods

### Strains and Media

Mycobacterial strains, including 29 ATCC reference strains (Table 1) and 17 NTM clinical isolates (M. abscessus USFLJA0001-M. kansasii USFLJA0017) were obtained from the Microbiology Laboratory, Department of Health-Bureau of Laboratories, Jacksonville, Fla. They were cultured in either Middlebrook 7H9 broth or Lowenstein Jenson media at appropriate temperature and stored in −80° C. with 15% glycerol.

### Multiple Genome Comparison

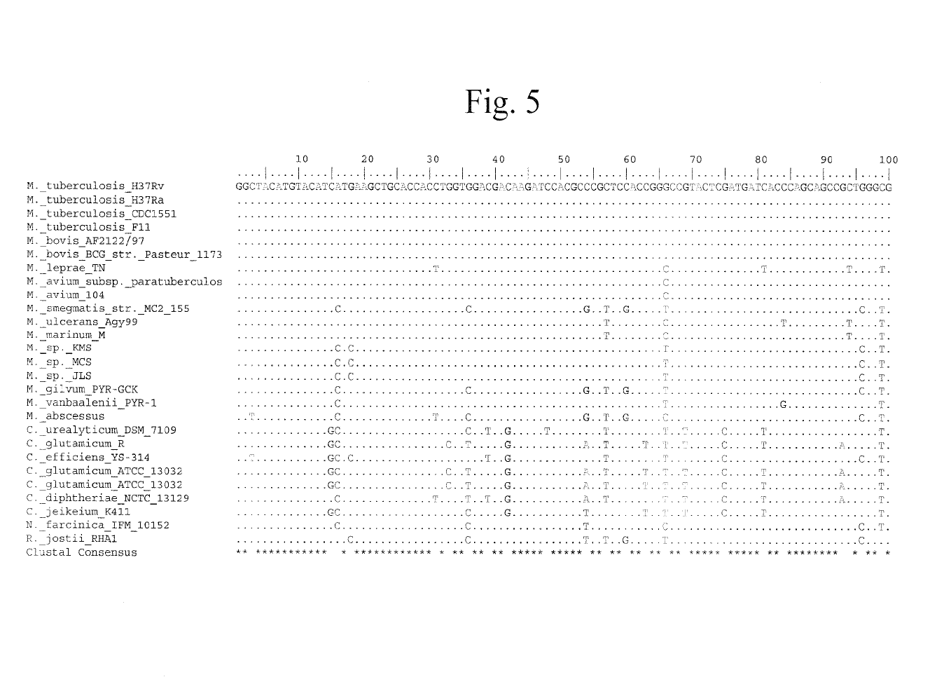

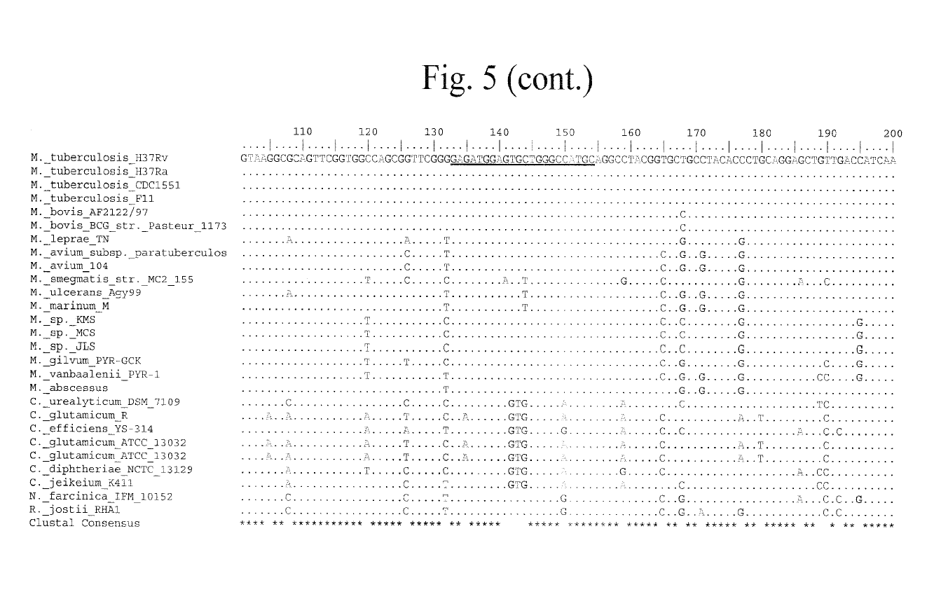

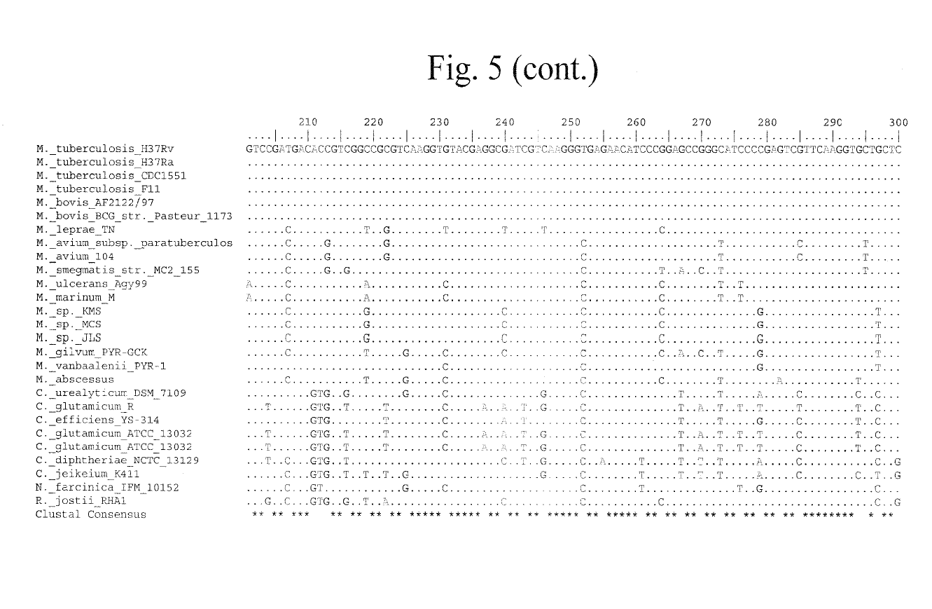

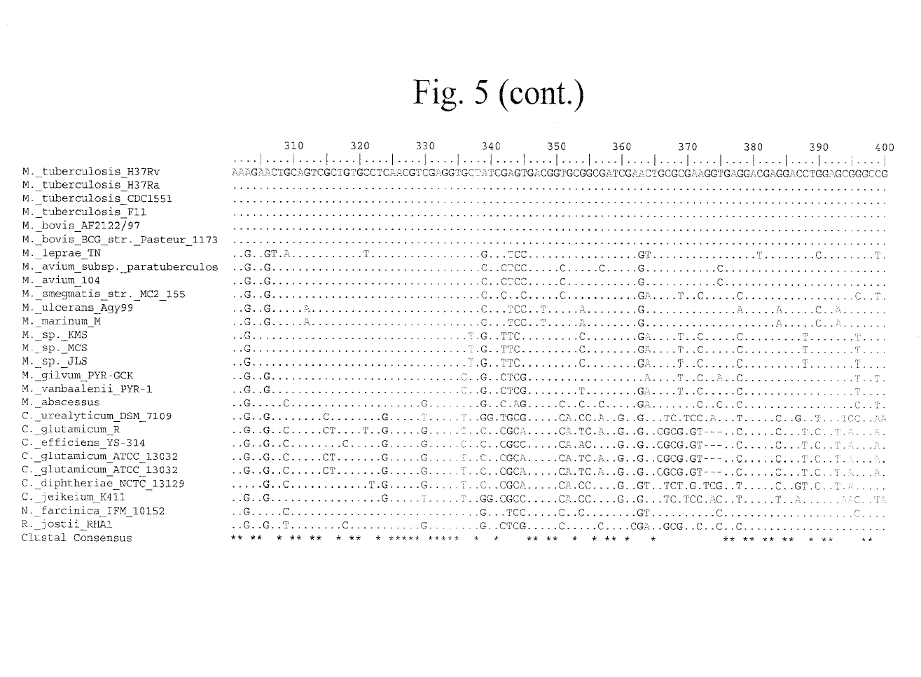

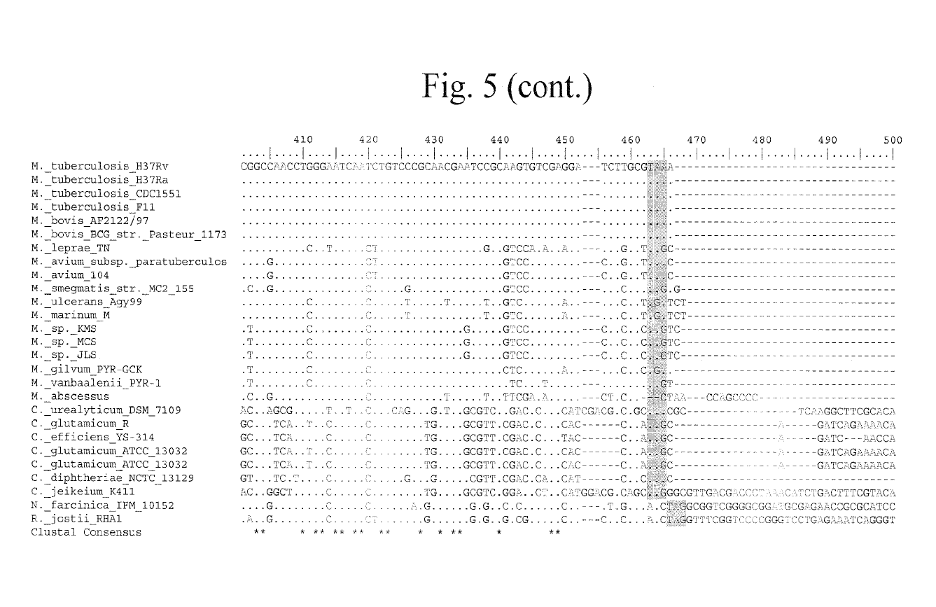

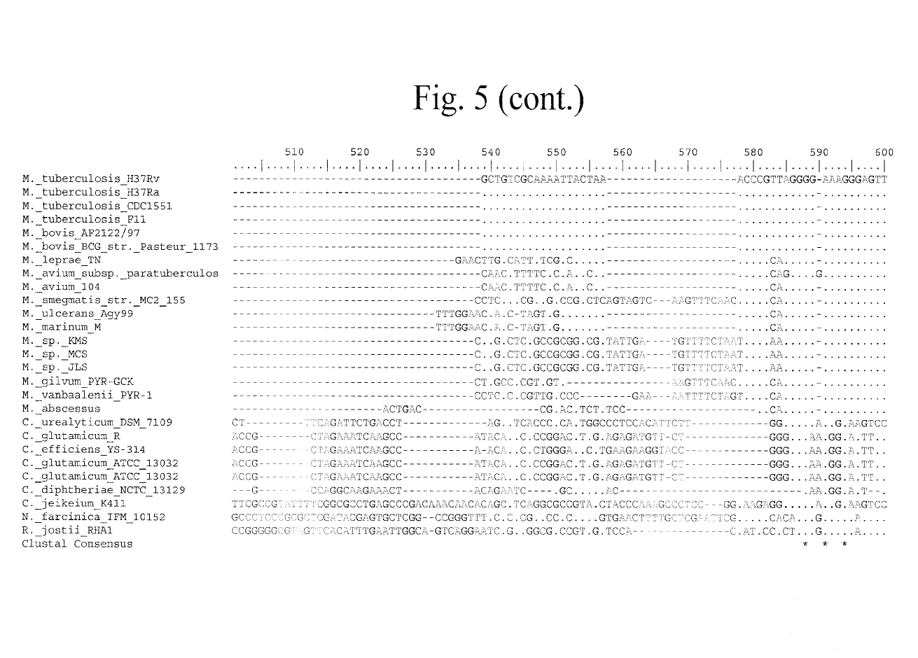

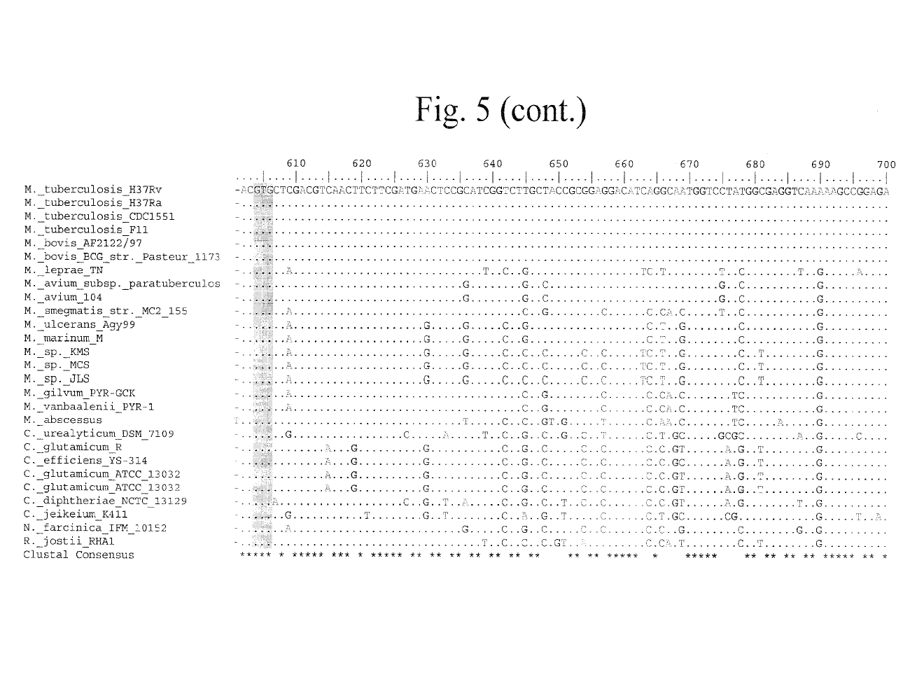

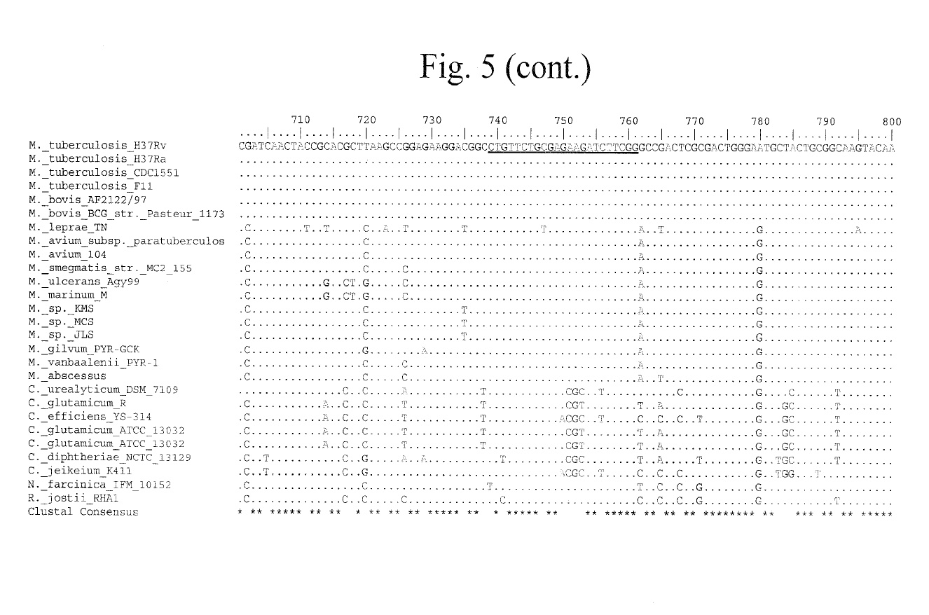

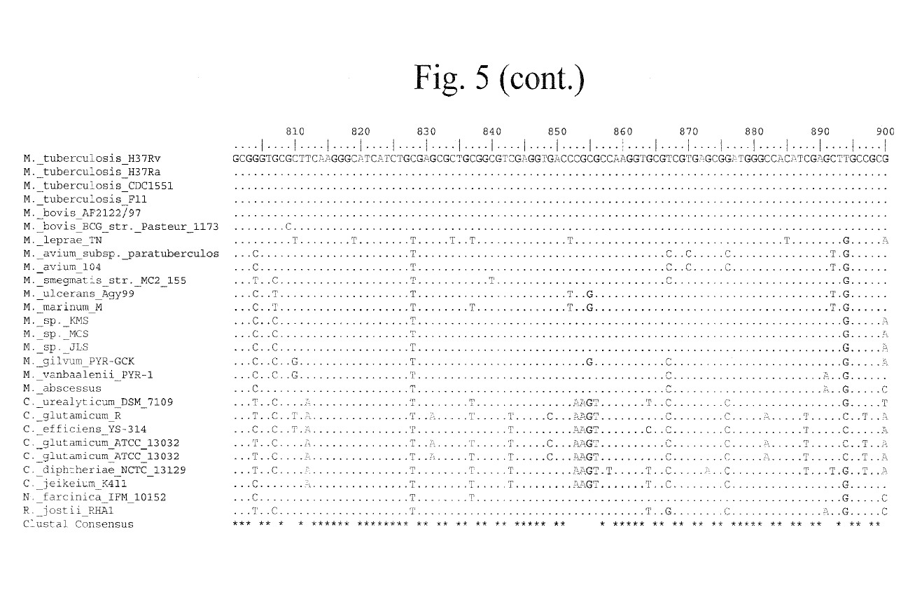

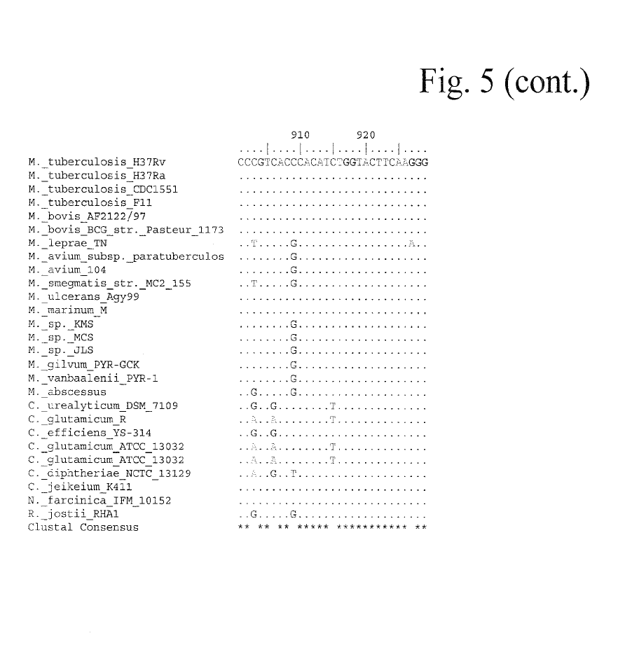

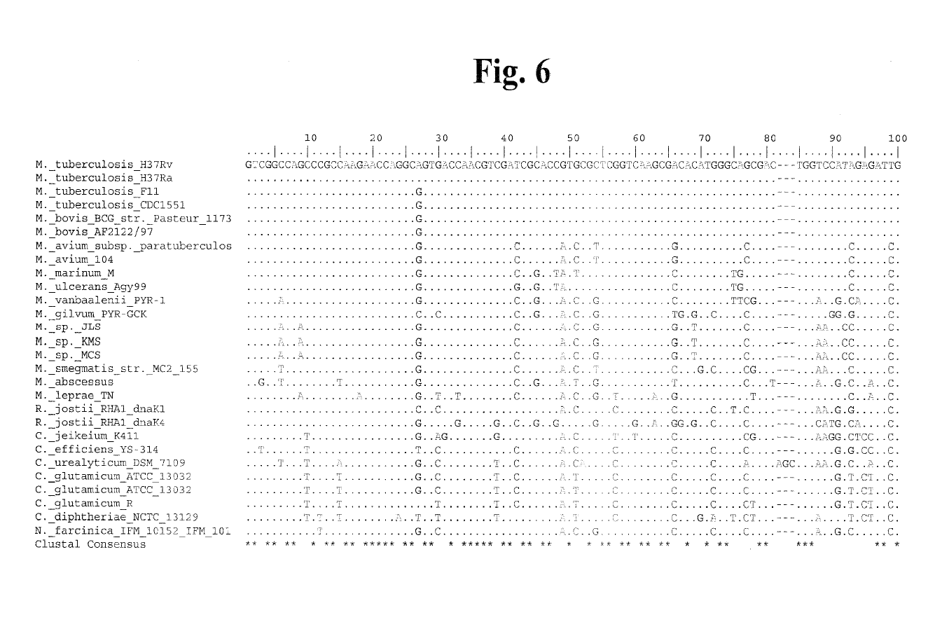

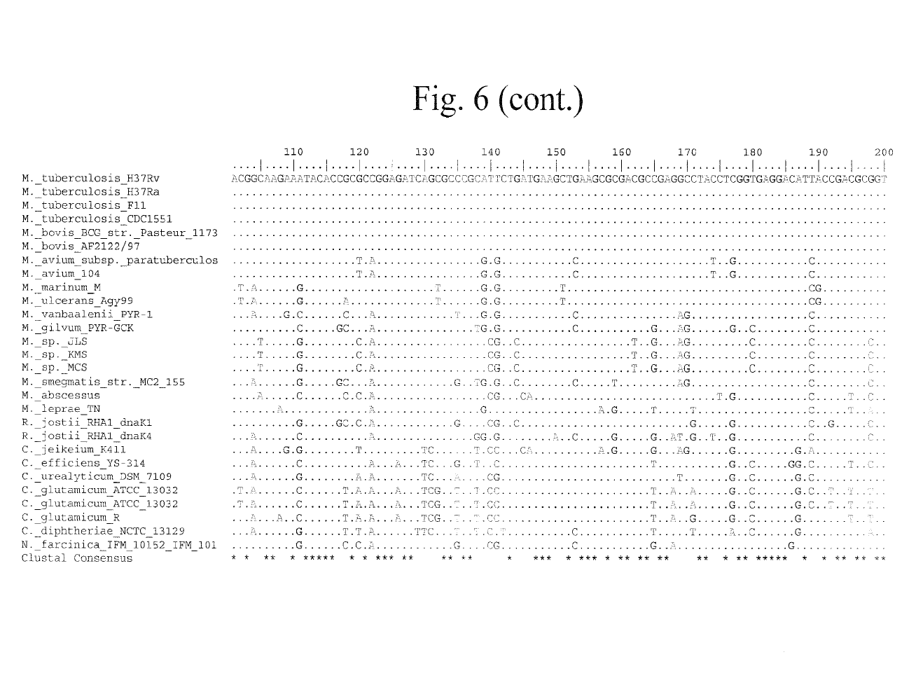

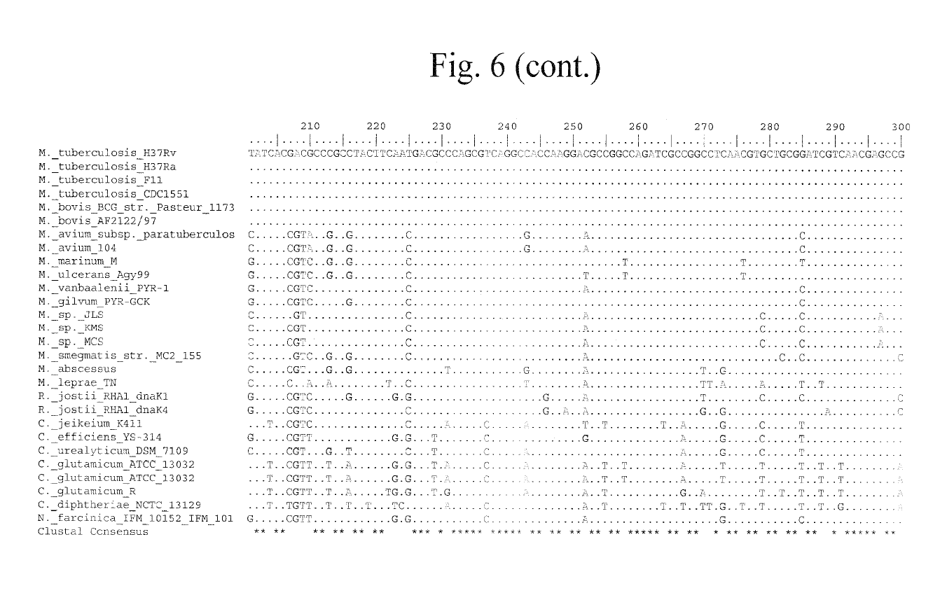

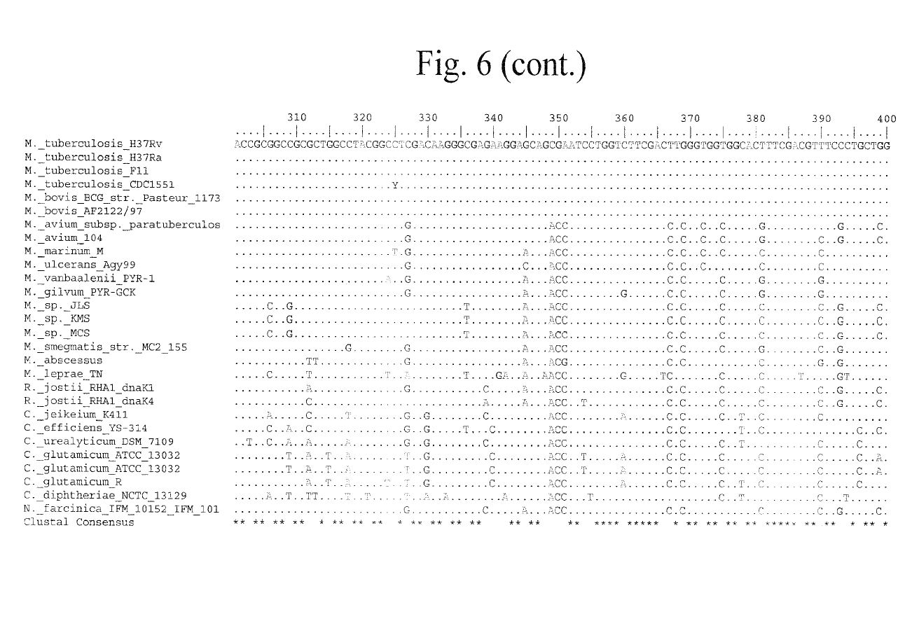

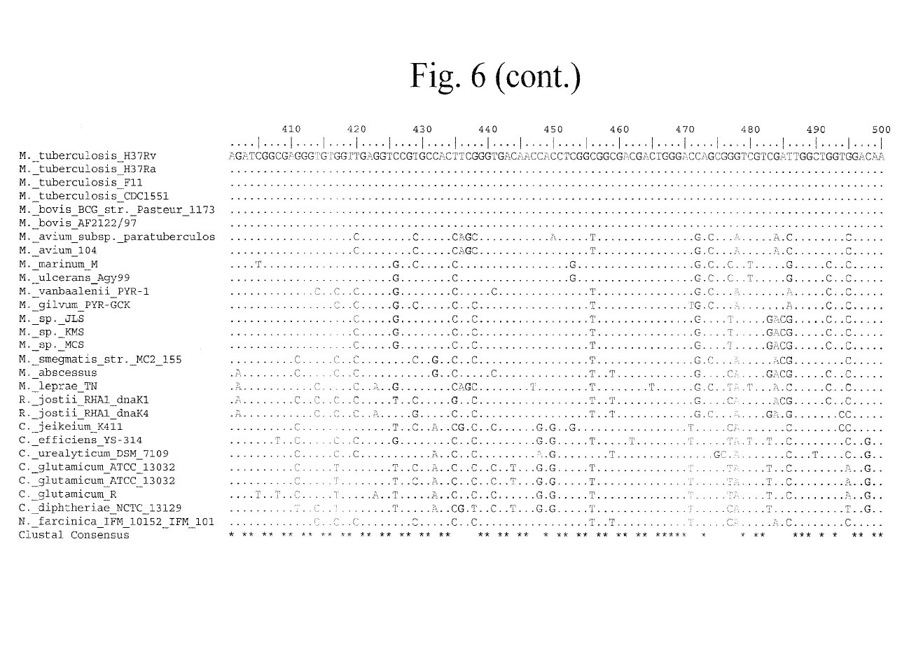

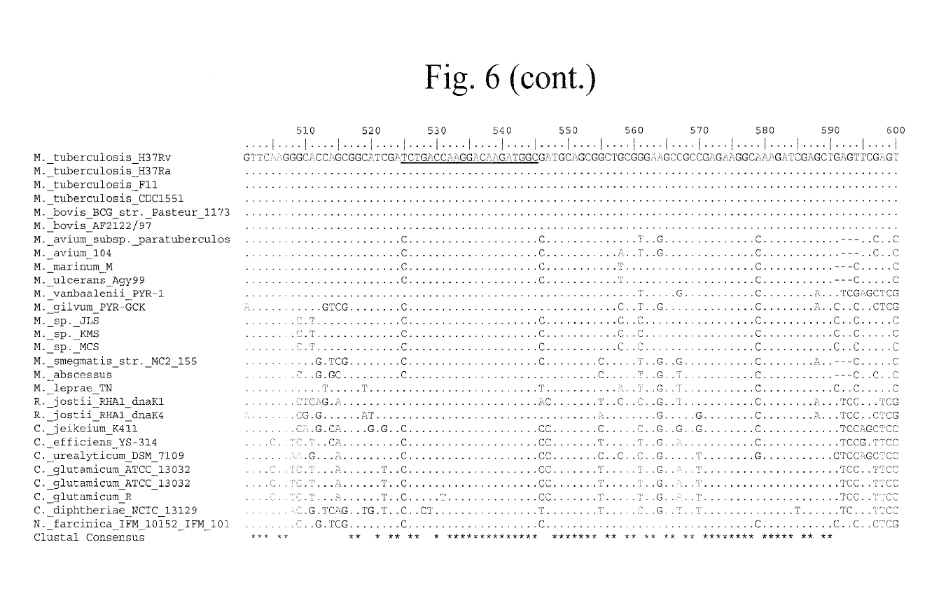

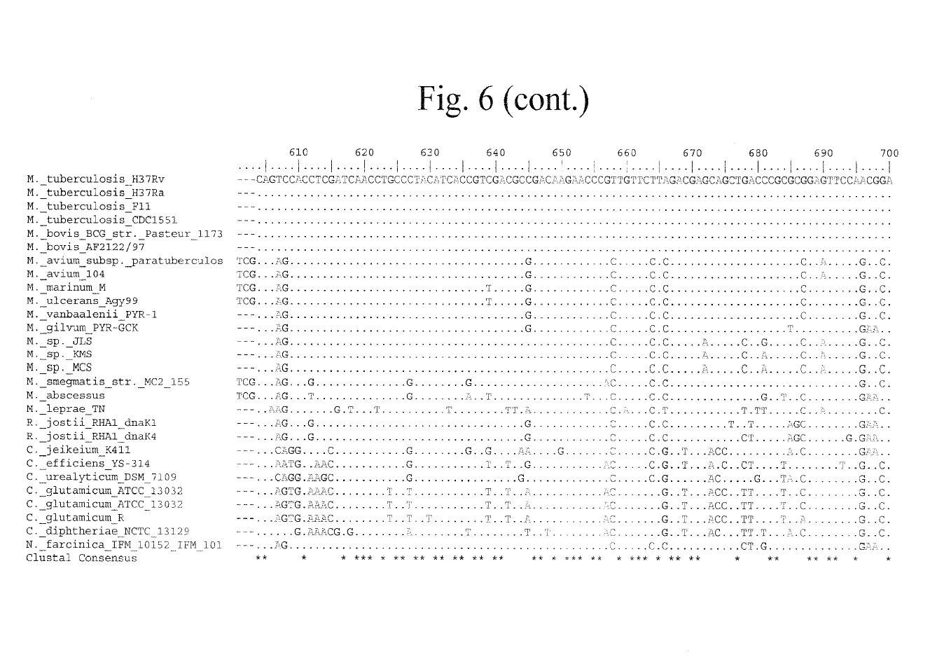

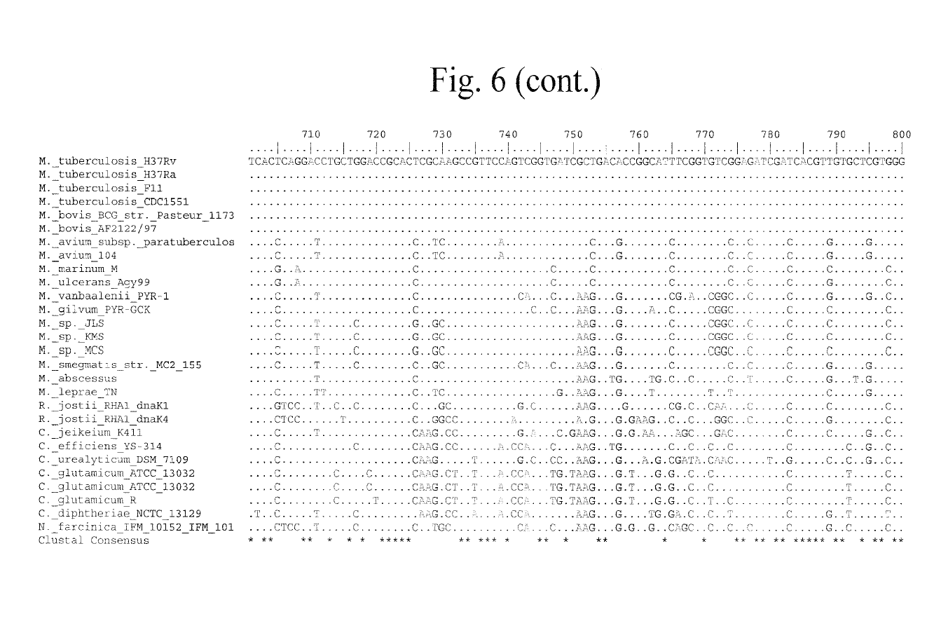

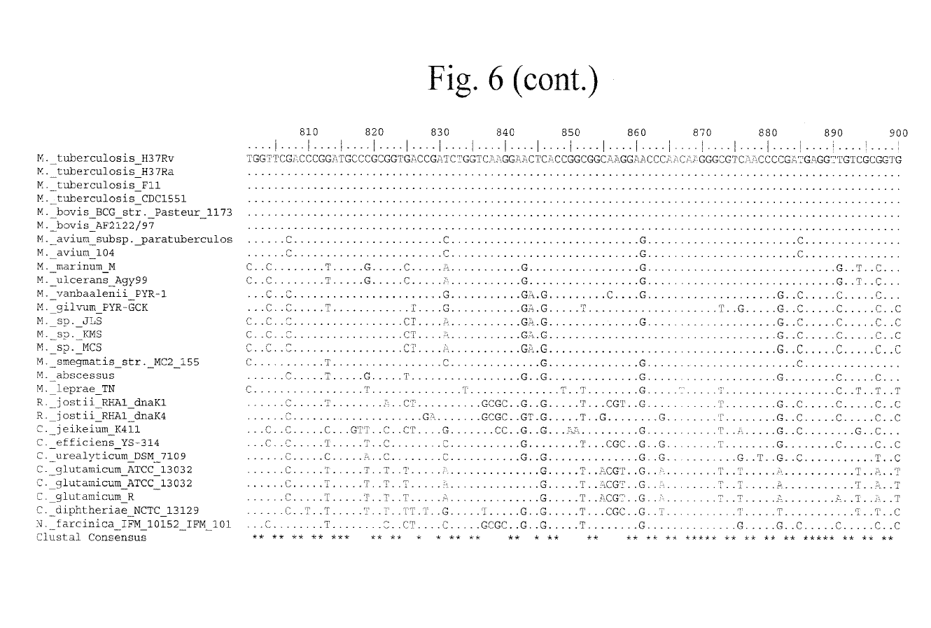

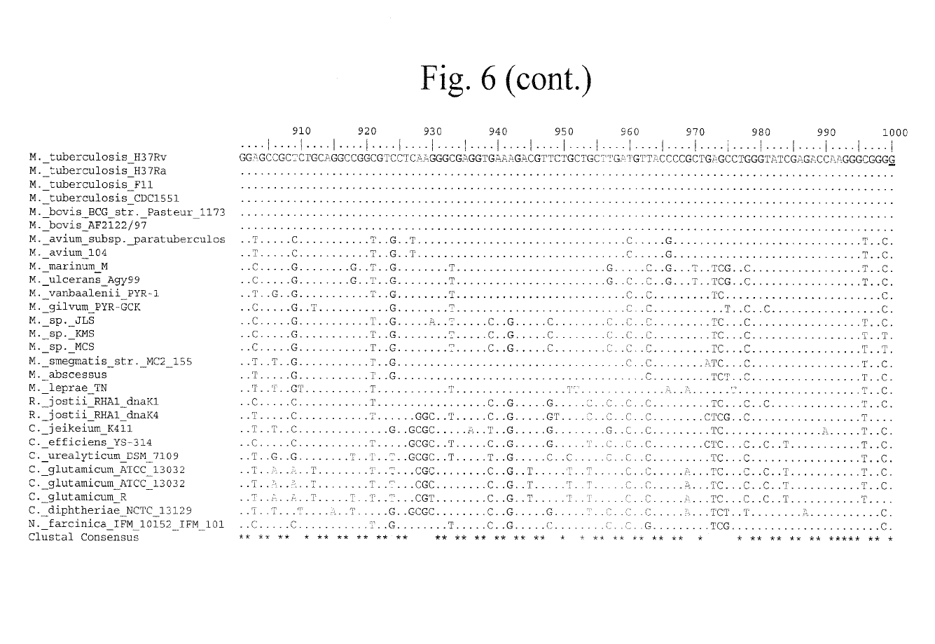

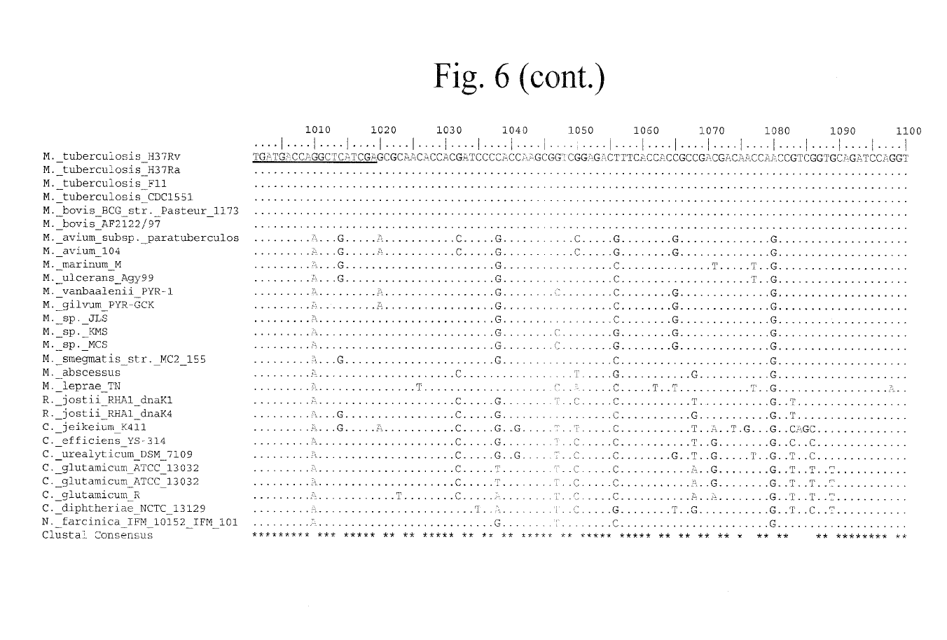

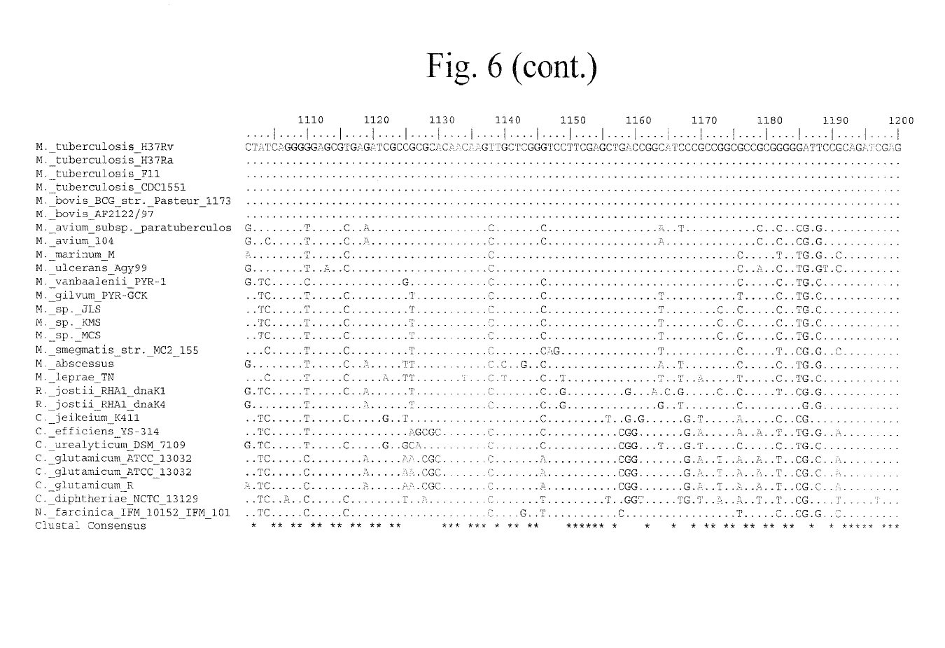

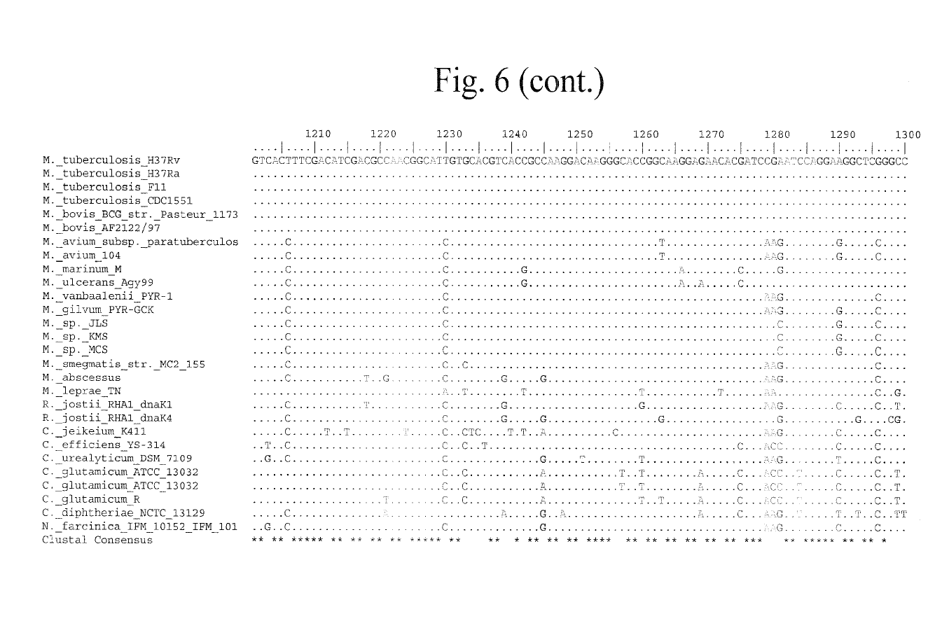

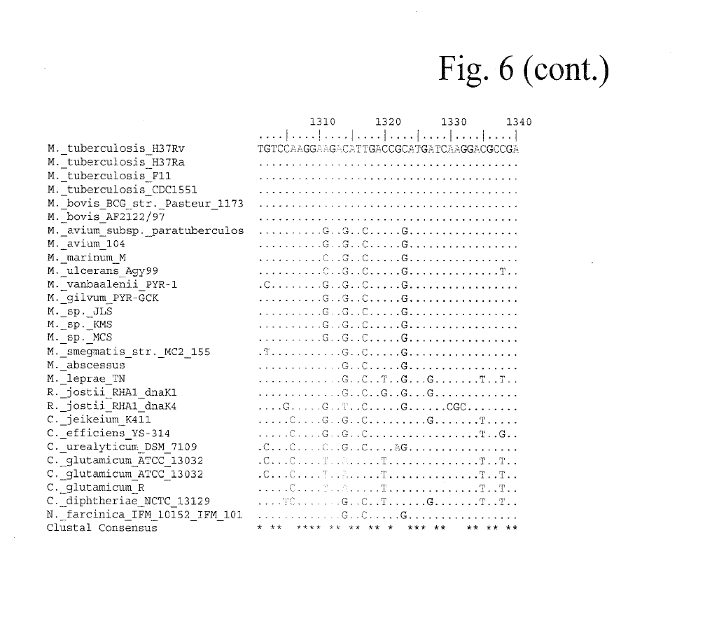

Eighteen mycobacterial genomes and nine genomes from eight closely related species in the suborder Corynebacterineae (Table 2) were used in a multi-genome comparison study to search for informative typing loci. Each genomic sequence was compared to the reference genome of M. tuberculosis H37Rv (GenBank Accession: NC_{—}000962) using BLASTN 2.2.18 running locally. Parameter “−m 8” was used to generate tabulated outputs of BLASTN and other options remained as default settings. These outputs from BLASTN were run through Perl scripts (available upon request) to extract the common homologous regions (CHRs) among these genomes. The CHRs are segments of DNA sequences that have BLASTN hits (>300 bp) in all 26 genome comparisons. They are marked with coordinates of the reference genome of M. tuberculosis H37Rv (Table 3). Amplification primers for typing loci were determined from the multiple sequence alignments of CHRs (FIGS. 5 and 6).

### DNA Extraction and Sequencing

Bacterial cells were spun down from 100 μl liquid media or scraped from solid media, then resuspended in 100 pal TE (10 mM Tris-HCl, 1 mM EDTA, pH 7.5). Suspensions were boiled for five minutes followed by centrifugation for two minutes and supernatants containing genomic DNA were collected and stored in −20° C. Two U of template DNA mixed with two μl of 1 mM MgCl₂ was used in each 20 μl PCR reaction that also contains 500 pM of each primers, 200 μM. of each dNTP, and 0.5 Unit of Taq DNA polymerase in 1× Buffer IV (Thermo Scientific, USA). The rpoBC loci were amplified using primer rpoBCF1 (5′-GAGATGGAGTGCTGGGCCATGC-3′) and primer rpoBCR1 (5′-CCGAAGATCTTCTCGCAGAACAG-3′) in the following PCR program: 95° C. for 1 min followed by 30 cycles of 95° C. 30 sec, 55° C. 30 sec, 72° C. 30 see, and ending with 72° C. for 2 min. Primers for dnaK locus arc dnaKFl (5′-CTGACCAAGGACAAGATGGC-3′) and dnaKR1 (5′-TCGATCAGCTTGGTCATCAC-3′). The PCR program for dnaK loci is the same as that for rpoBC except for using 50° C. as the annealing temperature. The hsp65 locus was amplified as previously reported (63). PCR products were purified with Qiagen MinuElute™ 96 UF PCR Purification Kit and sequenced from both ends with the same amplification primers. Nucleotide sequences were assembled using the phredPhrap software package (14, 15, 21).

### Phylogenetic Analysis

Sequences of all CHRs were extracted from the 18 completed mycobacterial genomes and the genome of Nocardia farcinica IFM 10152. Concatenations of these sequences were aligned and analyzed in MEGA4.1beta (33) with Neighbor-Joining method bootstrapped with 2000 replicates. The Maximum Composite Likelihood method with Complete deletion option for gaps was used to calculate the evolutionary distances. The Sequence from the Nocardia farcinica IFM 10152 genome was used to provide root for the phylogenetic tree.

Nucleotide sequences of dnaK, hsp65, and rpoBC loci from the collection of 46 reference and clinical strains (Table 1) were determined (Genbank accessions listed in Table 6). Three of the 29 ATCC strains in Table 1 have their genomic sequences available and sequencing results of dnaK, hsp65, and rpoBC loci in this study are identical to those published sequences. Phylogenetic tree of each individual locus and their concatenated sequence were constructed as mentioned above except that the pairwise deletion option was used for gaps due to many gaps in the multiple sequence alignment of rpoBC locus. Congruencies among trees were analyzed by program Conscnsc from the Phylip-3.69 package (www.evolution.genetics.washington.edu/phylip.html).

### Results

### Multiple Genome Comparison Inferred Evolution Relations Among Mycobacterium Species

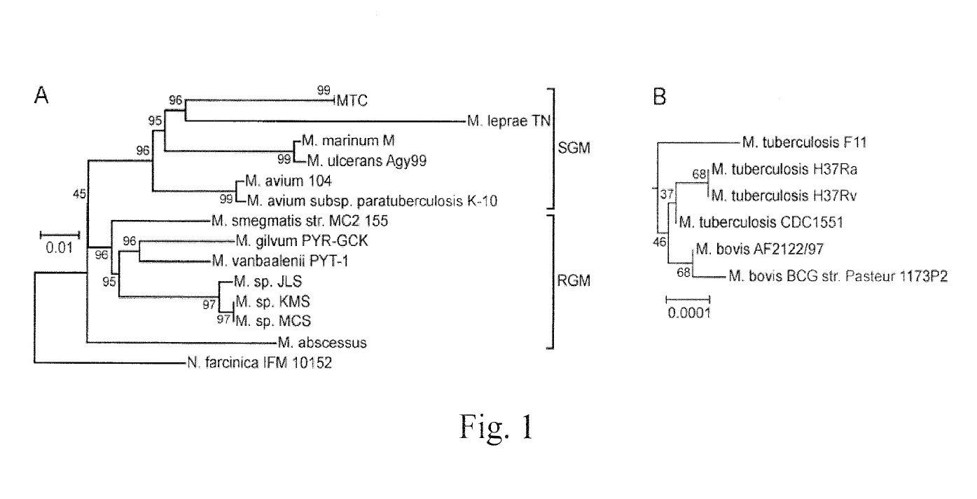

Our multiple genome comparison study of 27 genomes (Table 2) in the suborder Corynebacterineae has identified 26 CHRs which are potential loci for typing Mycobacterium (Table 3). These CHRs are highly conserved among species of the genera Mycobacterium, Corynebacterium, Nocardia, and Rhodococcus. The concatenated sequences of these 26 CHRs from the 18 mycobacterial genomes range from 13689 to 13,708 bp and cover 17 genes. The length differences are due to the gaps in the non-protein-coding regions, such as the intergenic region between EF-Tu and EF-G genes in the M. abscessus genome and in the ribosomal RNA operons of some mycobacteria. Sequence from N. farcinica IFM 10152 was used as an outgroup to construct a rooted tree. The phylogeny built upon these 26 CHRs is very robust and discriminative (FIG. 1Λ). More than 66% (10 out of 15) nodes are supported by >95% bootstrap values. It separates slow growing mycobacteria from rapidly growing ones and is even able to differentiate strains within the MTC cluster (FIG. 1B). M. sp. KMS and M. sp. MCS are very closely related and have identical sequences at these 26 CHRs.

Informative Loci for Typing were Identified from Common Homologous Regions

Currently, it is impractical to use whole genome sequences or 26 separate loci to differentiate species. Using one or several housekeeping genes is much cheaper and easier for clinical and research laboratories. The majority of the genes previously used for typing can be found in our CHR list, validating this bioinformatic approach. Further study of these CHRs individually will provide more useful typing loci for species identification. Like those already been widely used gyrB, hsp65 (groEL), and 16S rDNA (rrs), a single CHR can be used as a typing locus. But combining two adjacent CHRs into one locus can take advantage of the non-homologous region between them thus giving more differentiation power in phylogenetic analysis. On the list of CHRs, we noticed that there were small gaps between the two CHRs within dnaK and between the last CHR in rpoB and the first one in rpoC (181 bp and 170 bp respectively) and we test both loci (designated as dnaK and rpoBC) on our collection of mycobacteria. Results indicated that they are excellent loci for typing mycobacterial species with great differential power and robustness.

The rpoBC Locus is a Robust Typing Locus and with Good Differentiation Power

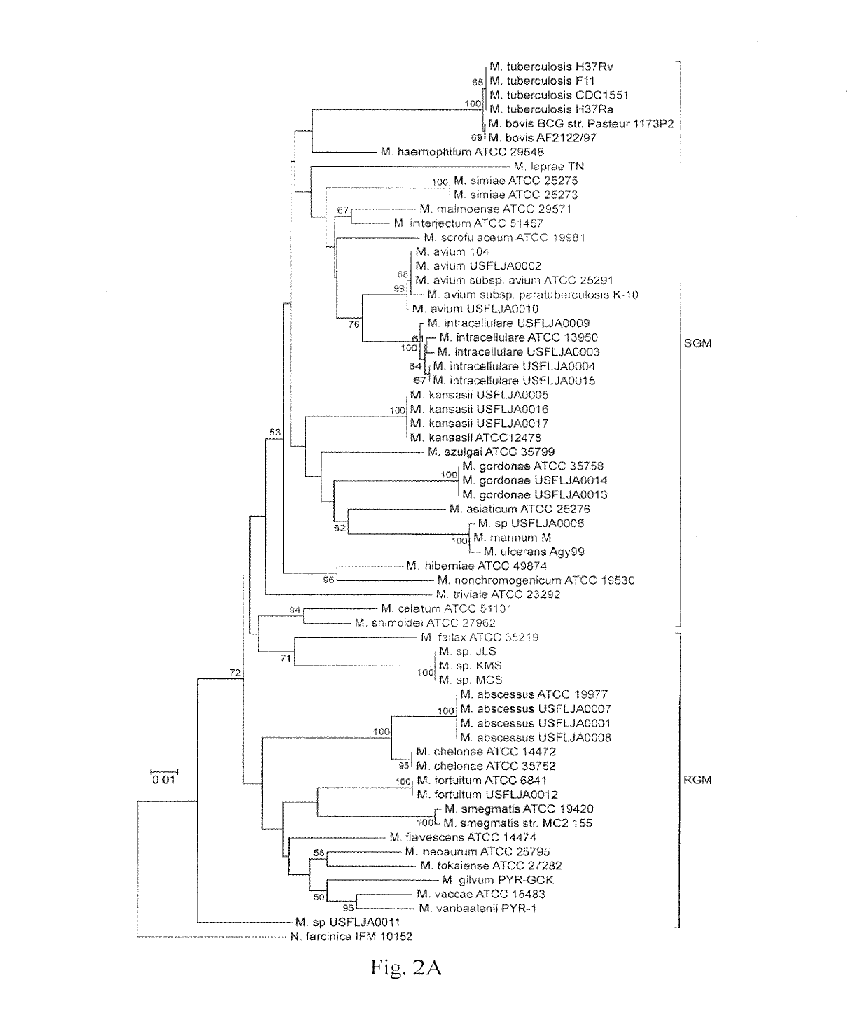

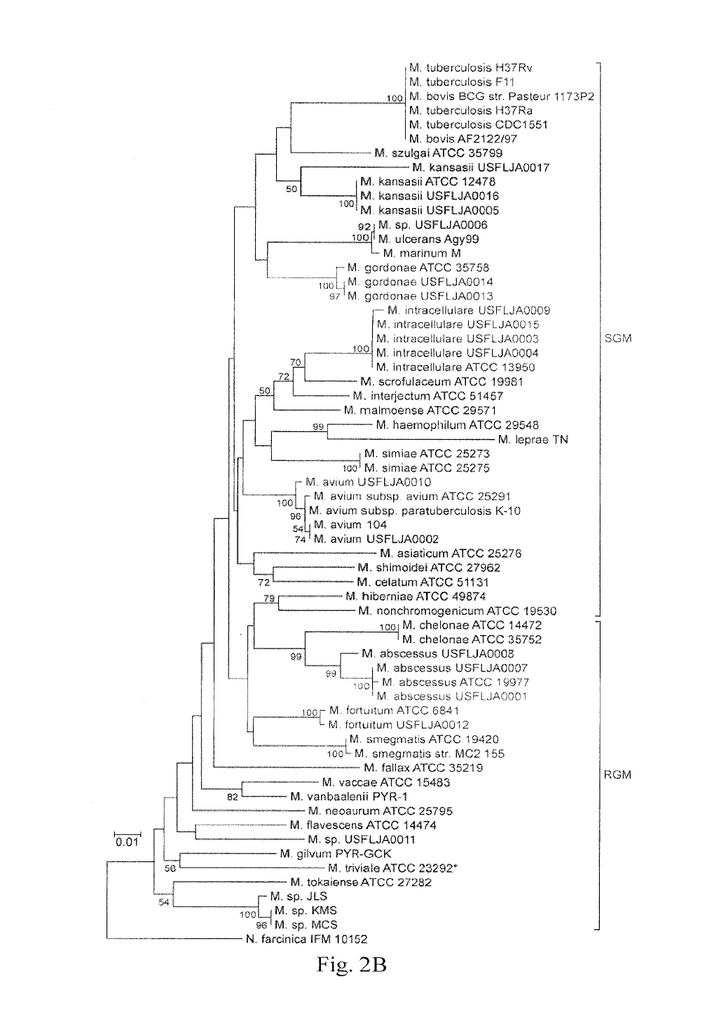

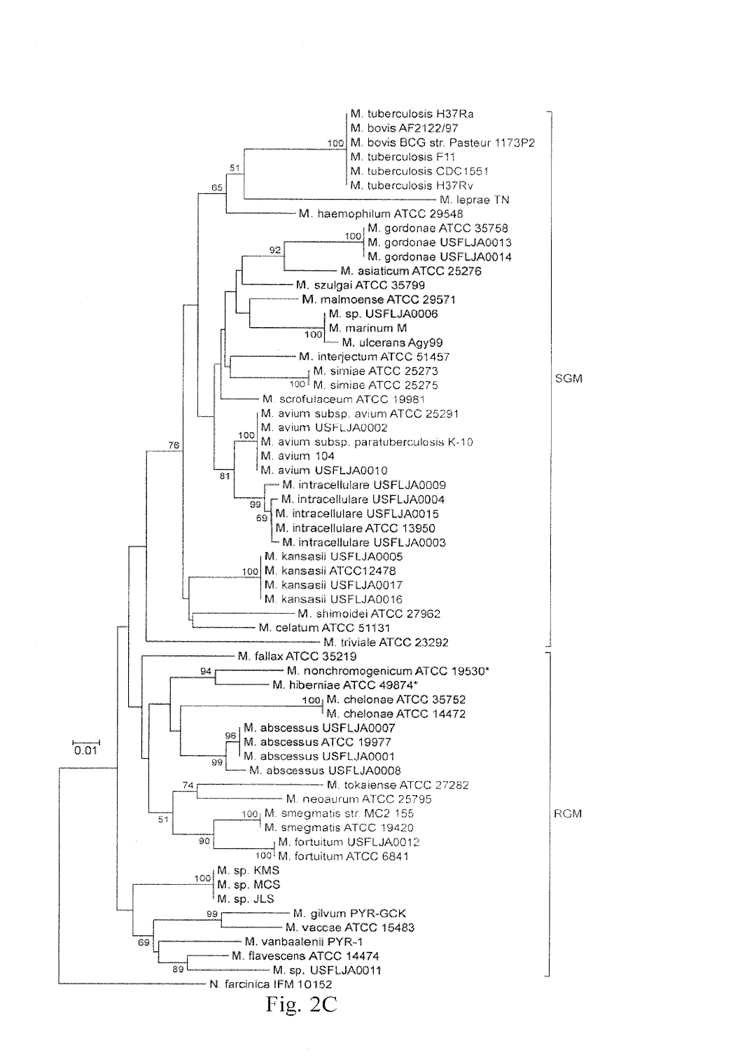

The rpoB and rpoC genes, encoding the β and the β′ subunits of the bacterial RNA polymerases respectively, are essential genes. The rpoBC locus, which covers portions of both rpoB and rpoC coding regions as well as the intergenic region between them, are easily amplified from flanking homologous regions in all tested mycobacterial species. Sequences from rpoBC range from 478 bp in the two M. chelonae species, M. celatum ATCC 51131, M. flavescens ATCC 14474, M. shimoidei ATCC 27962, and M. tokaiense ATCC 27282 to 510 bp in M. asiaticum ATCC 25276. It starts with the last 308 bp of rpoB and ends with the first 135 bp of rpoC coding regions. The length variability is solely due to the differences in the intergenes (FIG. 5). The intergenes are so variable that it is impossible to alignment them without the anchoring from the flanking rpoB and rpoC CHRs and a lot of gaps are left in the intergenic region of the multi-sequence alignment of this locus. Thus, “Pairwise Deletion” of gaps and Maximum Composite Likelihood method were used in phylogeny analysis (FIG. 2A). The mean distance at the rpoBC locus among the 61 mycobacterial strains is 0.118 (0.095 for hsp65) with the maxima of 0.179 from comparisons of M. leprae TN to M. abscessus strains (0.192 for hsp65 from comparison of M. leprae TN to M. gilvum PYR-GCK). Of all 60 nodes, 18 (30%) have bootstrap values greater than 75%, and 30 (50%) greater than 50%. In comparison, hsp65 has 20 (33%) and 26 (43%) respectively. The robust rpoBC locus also has great differentiation power. It not only differentiates the strains within the M. avium clade, the M. intracellulare clade, and the two M. smegmatis strains, but also separate M. tuberculosis (Biosafety Level 3) from M. bovis (Biosafety Level 2) which almost all other typing loci have failed (Table 5). Unlike hsp65 which put slow growing M. hiberniae and M. nonchromogenicum into RGM clade, the rapidly and slow growing groups are clearly separated in rpoBC tree. The clinical isolates except M. sp. USFLJA0011 were clustered with one of the typed strains, providing the clear identification of these isolates. M. sp. USFLJA0011 is placed outside of the mycobacterium clade in the rpoBC phylogenetic tree.

The dnaK Locus Provides Great Differential Power for Typing

Like hsp65, dnaK is a housekeeping gene, encoding another heatshock protein, Hsp70. Both of them are highly conserved among almost all organisms. They facilitate the folding of intercellular proteins and prevent protein aggregation which is highly toxic to cell function (reviewed in (71)). The dnaK gene has been used for typing in Brucella (10), Ochrobactrum (64), Xanthomonas (72), Clostridium (45), and some nitrogen-fixing genus (38, 44, 69). In mycobacteria, we identified a 451 bp fragment as the dnaK locus (alignment available in FIG. 6). The Neighbor-Joining phylogenetic tree of the dnaK locus is shown in FIG. 2B. The overall mean distance of this locus is 0.100 with maxima from of 0.195 from M. leprae TN vs. M. tokaiense ATCC 27282. Thirty-five percent (21 out of 60) of the nodes are supported by >75% bootstrap values and 51.7% nodes by >50% bootstrap values. The dnaK locus is the most robust among the three loci studied here. The dnaK locus also shows very good differential power and provides even more details than the rpoBC locus in some clusters such as M. avium, M. gordonae, M. fortuitum, M. kansasii, and M. abscessus (Table 5). It also partially differentiates the tree polycyclic aromatic hydrocarbon-degrading Mycobacterium isolates (JLS, KMS, and MCS) from the same superfund site (42). This separation is only observed in the phylogenetic analysis using 26 CHRs. But, it fails to differentiate species in MTC, and the resolution in M. intracellulare and M. smegmatis is lower than hsp65 and rpoBC. The division between RGM and SGM is not as clear as that from rpoBC. The slow growing M. triviale is clustered with RUM. Both hsp65 and dnaK congruently place M. sp. USFLJA0011 adjacent to M. flavescens though supported by different bootstrap values (<50% for dnaK and 89% for hsp65).

Multilocus Sequence Analysis of Concatenated of dnaK, hsp65, and rpoBC Loci

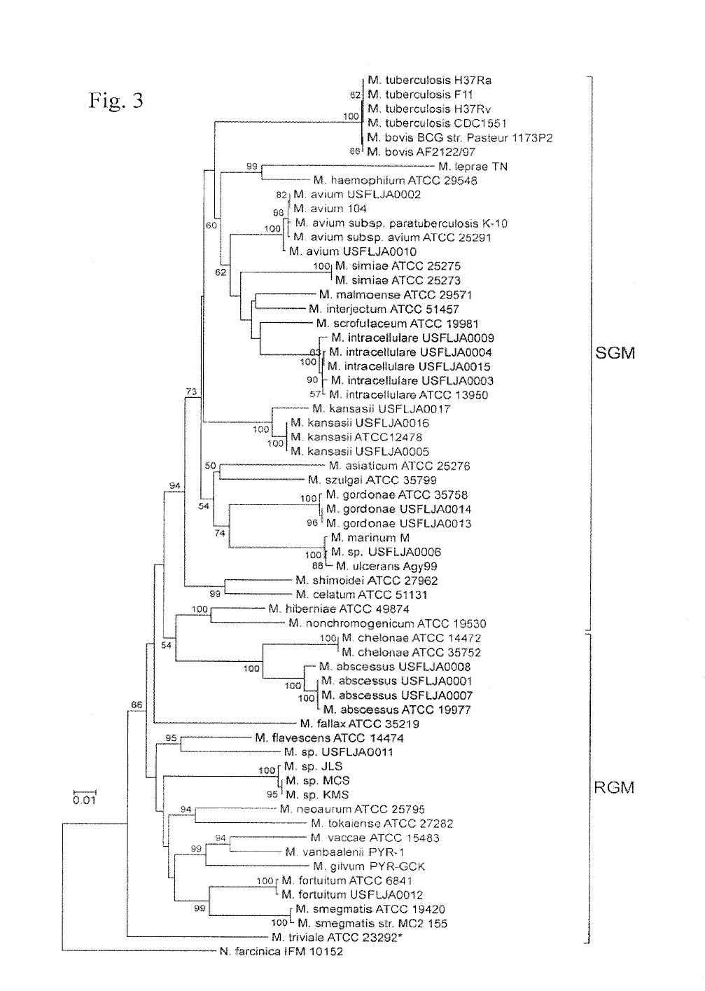

As we have seen in the three loci above as well as in other reports, the discrimination power of a single locus is limited and sometimes incorrect phylogeny is inferred. Concatenation of multiple loci combines the discriminative power from each locus. Congruent loci also provide a consensus evolutionary relationship among species, thus much more accurate. With a good congruency among dnaK, hsp65, and rpoBC (30% nodes are supported by phylogenies from all three loci), we have concatenated their sequences (more than 1330 bp) for a phylogenetic analysis (FIG. 3). This multilocus sequence analysis not only maintains the detailed separation in clusters such as MTC, M. avium group, M. intracellulare group, M. abscessus group, and the three polycyclic aromatic hydrocarbon-degrading mycobacteria, but also provides higher confidence (higher bootstrap values) than any single locus. Thirty-one nodes (51.6%) have bootstrap value >75% and 43 nodes (71.7%) with >50% bootstrap value. The separation between SGM and RGM is also good, except for M. triviale which also have usually been misplaced. M. sp. USFLJA0011 is clustered with M. flavescens with long splitting branches and a high bootstrap value, indicating that it probably belongs to another related species.

### Discussion

We have systematically compared the genomes from the suborder Corynebacterineae to locate 26 potential genomic regions for typing mycobacteria. Phylogenetic analysis of these 26 regions has inferred the evolutionary relations among mycobacterium species. The analysis provides more evidence that M. tuberculosis is the ancestor of M. bovis and the derivation of M. bovis BCG from M. bovis which is also supported by phylogenetic analysis on deleted regions (43).

From these 26 CHRs, we further selected four adjacent CHRs and combined them into two loci, dnaK and rpoBC, for typing mycobacterial strains. Results were compared to the commonly used locus, hsp65. Both new loci show greater discrimination power and provide valuable information for identification of mycobacterial species. As the first locus including intergenic region between two protein-coding genes and the second intergenic locus for typing mycobacteria (the other one is the ITS locus in rDNA operon), the rpoBC locus varies not only in its nucleotide sequence but also its length. It provides a good target for designing hybridization-based methods and size-differentiation-based methods to detect and identify mycobacterial species. The differentiation power of rpoBC in MTC also provides evolution information that agrees with the finding from the analysis of 26 CHRs. Besides M. tuberculosis and M. bovis, we also sequenced the rpoBC locus from another MTC member, M. microti ATCC 19422 (GenBank Accession GU362516), which has identical rpoBC sequence to those from M. bovis but differs from those from M. tuberculosis. This result further supports the recently proposed evolutionary scenario of MTC, in which M. tuberculosis is an ancestral species of M. bovis and M. microti (6). The consensus phylogenetic tree from rpoBC, dnaK, and hsp65 is even more robust. We suggest the inclusion of the rpoBC and the dnaK loci into a future MLST scheme for Mycobacterium.

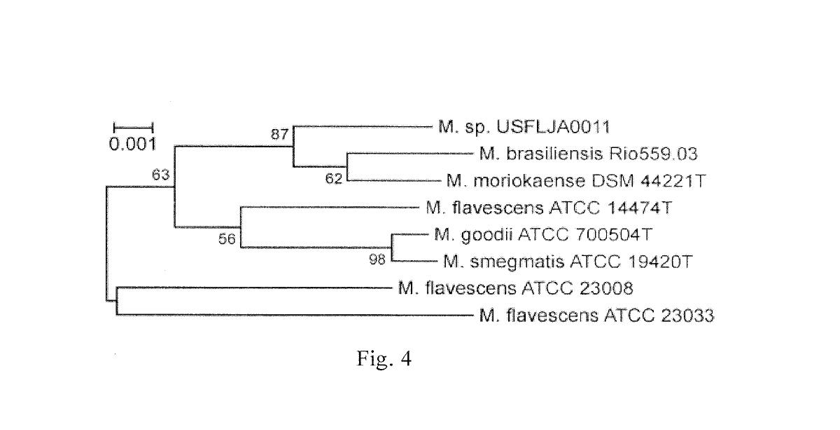

With our current strain collection for testing these new loci, we were able to associate most of our clinical isolates with typed strains except M. sp. USF LJA0006 and M. sp. USFLJA0011. This indicates that these loci are very useful in diagnosis of mycobacterial infections. M. sp. USFLJA0006 unambiguously belongs to M. marinum-M. ulcerans group. But it is difficult to assign species identity due to the great sequence similarity between M. marinum and M. ulcerans. The other clinical isolate, M. sp. USF LJA0011, is a rapidly growing NTM with yellow colonies. It was first identified as a strain of M. flavescens by hsp65 RFLP and it is related to M. flavescens in both hsp65 and dnaK alignments. But rpoBC locus reveals the discrepancy. M. sp. USFLJA0011 is placed as an outgroup of mycobacteria. The BLASTN result of the 1446 bp 16S rDNA sequence of M. sp. USFLJA0011 (GenBank Accession GU362538) has indicated that it is closest to “M. brasiliensis” strain Rio559.03 (Genbank Accession EU165538) (35) with 99.2% (1435/1446) identity including one gap. “M. brasiliensis” is not an accepted species when this paper was written. The closest typed strain is nonphotochromogenic M. moriokaense CIP 105393 (GenBank Accession AY859686) with 99.2% (1434/1446) identity and no gap, but their colony morphologies differ. The 16S rDNA sequence of M sp. USFLJA0011 is quite distant from the typed M. flavescens strain ATCC 14474 (1418/1446 identities with 2 gaps). Thus, it is likely to belong to a new species of mycobacteria. We also compared it with two other M. flavescens strains ATCC 23008 and ATCC 23033 whose 16S rDNA sequences are available in GenBank. Our result showed that they were even farther from M. sp. USFLJA0011 than the typed M. flavescens ATCC 14474. The similarities among these three M. flavescens 16S rDNA sequences are even lower than the interspecies similarities among M. goodie, M. smegmatis, M. moriokaense, and M. flavescens, as seen in earlier reports (65, 74) (FIG. 4). Their nomenclatures need to be reconsidered.

M. nonchromogenicum and M. hiberniae are slow growing mycobacteria. They belong to the M. terrae complex which is frequently placed into the clade of RGM or between the RGM and SGM in the phylogenetic analysis of other loci. Associated with them in our analysis are RUM M. abscessus and M. chelonae. Another SGM, M. celatum is also close to the RGM border. Interestingly, these species are exceptions to the general rule that RGM have two identical rDNA operons while SGM have only one (1). For example, M. terrae and M. celatum have been reported to containing two different rDNA operons (47, 50) while M. abscessus and M. chelonae genomes contain only one rDNA operon. It is possible that these species are the intermediate transition species between SGM and RGM.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

TABLE 1
Mycobacterial strains included in this study.

M. abscessus ATCC 19977 (M. abscessus, genome GenBank
Accession: NC_010397)
M. asiaticum ATCC 25276
M. avium subsp. avium ATCC 25291
M. celatum ATCC 51131
M. chelonae ATCC 14472
M. chelonae ATCC 35752
M. fallax ATCC 35219
M. flavescens ATCC 14474
M. fortuitum ATCC 6841
M. gordonae ATCC 35758
M. haemophilum ATCC 29548
M. hiberniae ATCC 49874
M. interjectum ATCC 51457
M. intracellulare ATCC 13950
M. kansasii ATCC 12478
M. malmoense ATCC 29571
M. marinum ATCC BAA-535 (M. marinum M, genome GenBank
Accession: NC_010612)
M. neoaurum ATCC 25795
M. nonchromogenicum ATCC 19530
M. scrofulaceum ATCC 19981
M. shimoidei ATCC 27962
M. simiae ATCC 25273
M. simiae ATCC 25275
M. smegmatis ATCC 19420
M. szulgai ATCC 35799
M. tokaiense ATCC 27282
M. triviale ATCC 23292
M. tuberculosis ATCC 27294 (M. tuberculosis H37Rv, genome GenBank
Accession: NC_000962)
M. vaccae ATCC 15483
M. abscessus USFLJA0001
M. avium USFLJA0002
M. intracellulare USFLJA0003
M. intracellulare USFLJA0004
M. kansasii USFLJA0005
M. sp. USFLJA0006
M. abscessus USFLJA0007
M. abscessus USFLJA0008
M. intracellulare USFLJA0009
M. avium USFLJA0010
M. sp. USFLJA0011
M. fortuitum USFLJA0012
M. gordonae USFLJA0013
M. gordonae USFLJA0014
M. intracellulare USFLJA0015
M. kansasii USFLJA0016
M. kansasii USFLJA0017

TABLE 2
The completed genomes used in multiple genome comparison.

| Genbank Accession | Strain | Reference |
|---|---|---|
| NC_010397 | Mycobacterium abscessus | |
| NC_008595 | Mycobacterium avium 104 | |
| NC_002944 | Mycobacterium avium subsp. | (37) |
| | paratuberculosis K-10 | |
| NC_002945 | Mycobacterium bovis AF2122/97 | (19) |
| NC_008769 | Mycobacterium bovis BCG | (5) |
| | str. Pasteur 1173P2 | |
| NC_009338 | Mycobacterium gilvum PYR-GCK | |
| NC_002677 | Mycobacterium leprae TN | (12) |
| NC_010612 | Mycobacterium marinum M | (58) |
| NC_008596 | Mycobacterium smegmatis str. | |
| | MC2 155 | |
| NC_009077 | Mycobacterium sp. JLS | |
| NC_008705 | Mycobacterium sp. KMS | |
| NC_008146 | Mycobacterium sp. MCS | |
| NC_002755 | Mycobacterium tuberculosis CDC1551 | (17) |
| NC_009565 | Mycobacterium tuberculosis F11 | |
| NC_009525 | Mycobacterium tuberculosis H37Ra | (75) |
| NC_000962 | Mycobacterium tuberculosis H37Rv | (11) |
| NC_008611 | Mycobacterium ulcerans Agy99 | (59) |
| NC_008726 | Mycobacterium vanbaalenii PYR-1 | |
| NC_002935 | Corynebacterium diphtherias NCTC | (9) |
| | 13129 | |
| NC_004369 | Corynebacterium efficiens YS-314 | (48) |
| NC_006958 | Corynebacterium glutamicum ATCC | (27) |
| | 13032 | |
| NC_003450 | Corynebacterium glutamicum ATCC | (25) |
| | 13032 | |
| NC_009342 | Corynebacterium glutamicum R | (73) |
| NC_007164 | Corynebacterium jeikeium K411 | (61) |
| NC_010545 | Corynebacterium urealyticum DSM | (62) |
| | 7109 | |
| NC_006361 | Nocardia farcinica IFM 10152 | (26) |
| NC_008268 | Rhodococcus jostii. RHA1 | (39) |

TABLE 3
The 26 CHRs for potential typing loci on mycobacterial genomes.

| | | | Distance to next | |
|---|---|---|---|---|
| Start^{a} | End^{a} | Length (bp) | CHR^{c} (bp) | Gene name^{b} |
| 6650 | 6850 | 201 | 413132 | gyrB |
| 419982 | 420569 | 588 | 181 | dnaK |
| 420750 | 421315 | 566 | 38523 | dnaK |
| 459838 | 460166 | 329 | 69163 | clpB |
| 529329 | 529836 | 508 | 230400 | groEL |
| 760236 | 760562 | 327 | 327 | rpoB |
| 760889 | 761242 | 354 | 788 | rpoB |
| 762030 | 762571 | 542 | 293 | rpoB |
| 762864 | 763198 | 335 | 170 | rpoB |
| 763368 | 763695 | 328 | 629 | rpoC |
| 764324 | 765135 | 812 | 17385 | rpoC |
| 782520 | 782928 | 409 | 1874 | fusA1 |
| 784802 | 785188 | 387 | 396 | tuf |
| 785584 | 786002 | 419 | 14482 | tuf |
| 800484 | 800779 | 296 | 665726 | rpsJ |
| 1466505 | 1466847 | 343 | 5093 | atpD |
| 1471940 | 1473388 | 1449 | 1065 | rrs |
| 1474453 | 1474952 | 500 | 708 | rrl |
| 1475660 | 1476660 | 1001 | 356969 | rrl |
| 1833629 | 1834758 | 1130 | 1183953 | rpsA |
| 3018711 | 3019126 | 416 | 393014 | sigA |
| 3412140 | 3412485 | 346 | 633 | nrdE |
| 3413118 | 3413682 | 565 | 624998 | nrdE |
| 4038680 | 4039280 | 601 | 296 | clpC1 |
| 4039576 | 4040191 | 616 | 11707 | clpC1 |
| 4051898 | 4052235 | 338 | 359297 | fisH |

^{a}M. tuberculosis H37Rv genome coordinates are used.
^{b}M. tuberculosis H37Rv gene names are used.
^{c}Distances between adjacent CHRs are shown, with two small ones (less than 200 bp) in bold.

TABLE 4
Summary of loci features

| | Length | | Bootstrap >50% | Bootstrap >75% |
|---|---|---|---|---|
| Locus | (bp) | Mean distance | (%) | (%) |
| rpoBC | 478-510 | 0.118 | 30 | 50 |
| dnaK | 451 | 0.100 | 35 | 52 |
| hsp65 | 440 | 0.095 | 33 | 43 |

TABLE 5
Mean pairwise distances of dnaK, hsp65, and rpoB loci within
mycobacterial groups.

| Group | No. of strains | rpoBC | dnaK | hsp65 |
|---|---|---|---|---|
| Mycobacterium | 61 | 0.118 | 0.100 | 0.095 |
| M. abscessus | 4 | 0 | 0.0124 | 0.0044 |
| M. avium | 5 | 0.0025 | 0.0049 | 0 |
| MTC | 6 | 0.0011 | 0 | 0 |
| M. chelonae | 2 | 0 | 0 | 0 |
| M. fortuitum | 2 | 0 | 0.0045 | 0 |
| M. gordonae | 3 | 0 | 0.0045 | 0 |
| M. intracellulare | 5 | 0.005 | 0.0018 | 0.0035 |
| M. kansasii | 4 | 0 | 0.0366 | 0 |
| M. smegmatis | 2 | 0.004 | 0.0022 | 0 |

TABLE 6
GenBank Accession numbers of the dnaK, hsp65, and rpoBC loci used in
this study. The hsp65 loci sequences of several strains have same
sequences as previously deposited s. We list these accession numbers
instead.

| Organism | dnaK | hsp65¹ | rpoBC |
|---|---|---|---|
| M. asiaticum ATCC 25276 | GU362430 | GU362517 | GU362473 |
| M. avium subsp. avium | GU362431 | GQ153289 | GU362474 |
| ATCC 25291 | | | |
| M. celatum ATCC 51131 | GU362432 | AF547817 | GU362475 |
| M. chelonae ATCC 14472 | GU362433 | GU362518² | GU362476 |
| M. chelonae ATCC 35752 | GU362434 | AY458074 | GU362477 |
| | | AF547818 | |
| M. fallax ATCC 35219 | GU362435 | AF547829 | GU362478 |
| M. flavescens ATCC 14474 | GU362436 | GU362519³ | GU362479 |
| M. fortuitum ATCC 6841 | GU362437 | AY458072 | GU362480 |
| M. gordonae ATCC 35758 | GU362438 | AF547840 | GU362481 |
| M. haemophilum ATCC | GU362439 | GQ245967 | GU362482 |
| 29548 | | AF547841 | |
| M. hiberniae ATCC 49874 | GU362440 | AY438083 | GU362483 |
| M. interjectum ATCC 51457 | GU362441 | AF547846 | GU362484 |
| M. intracellulare ATCC | GU362442 | GQ153290 | GU362485 |
| 13950 | | DQ284774 | |
| | | AF126035⁴ | |
| M. kansasii ATCC12478 | GU362443 | AF434739 | GU362486 |
| | | AF547849 | |
| M. malmoense ATCC | GU362444 | GQ153293 | GU362487 |
| 29571 | | AF547854 | |
| M. neoaurum ATCC 25795 | GU362445 | AF547860 | GU362488 |
| M. nonchromogenicum ATCC | GU362446 | AF434732 | GU362489 |
| 19530 | | AF547861 | |
| M. scrofulaceum ATCC | GU362447 | GQ153288 | GU362490 |
| 19981 | | AF434733 | |
| | | AF547871 | |
| M. shimoidei ATCC 27962 | GU362448 | AF547874 | GU362491 |
| M. simiae ATCC 25273 | GU362449 | GU362520 | GU362492 |
| M. simiae ATCC 25275 | GU362450 | GQ153292 | GU362493 |
| | | AF434730 | |
| | | AF547875 | |
| M. smegmatis ATCC | GU362451 | AY458065 | GU362494 |
| 19420 | | AF547876 | |
| M. szulgai ATCC 35799 | GU362452 | AF547878⁵ | GU362495 |
| M. tokaiense ATCC 27282 | GU362453 | AF547881 | GU362496 |
| M. triviale ATCC 23292 | GU362454 | AF434737 | GU362497 |
| | | AF547883 | |
| M. vaccae ATCC 15483 | GU362455 | AF547889 | GU362498 |
| M. abscessus USFLJA0001 | GU362456 | GU362521 | GU362499 |
| M. avium USFLJA0002 | GU362457 | GU362522 | GU362500 |
| M. intracellulare | GU362458 | GU362523 | GU362501 |
| USFLJA0003 | | | |
| M. intracellulare | GU362459 | GU362524 | GU362502 |
| USFLJA0004 | | | |
| M. kansasii USFLJA0005 | GU362460 | GU362525 | GU362503 |
| M. sp. USFLJA0006 | GU362461 | GU362526 | GU362504 |
| M. abscessus USFLJA0007 | GU362462 | GU362527 | GU362505 |
| M. abscessus USFLJA0008 | GU362463 | GU362528 | GU362506 |
| M. avium USFLJA0009 | GU362464 | GU362529 | GU362507 |
| M. avium USFLJA0010 | GU362465 | GU362530 | GU362508 |
| M. sp. USFLJA0011 | GU362466 | GU362531 | GU362509 |
| M. fortuitum USFLJA0012 | GU362467 | GU362532 | GU362510 |
| M. gordonae USFLJA0013 | GU362468 | GU362533 | GU362511 |
| M. gordonae USFLJA0014 | GU362469 | GU362534 | GU362512 |
| M. intracellulare | GU362470 | GU362535 | GU362513 |
| USFLJA0015 | | | |
| M. kansasii USFLJA0016 | GU362471 | GU362536 | GU362514 |
| M. kansasii USFLJA0017 | GU362472 | GU362537 | GU362515 |

¹We have sequenced the hsp65 locus of all strains listed here. Since same sequences from same strains are already in GenBank, the available GenBank Accession numbers have been listed instead of submitting the sequences for new Accession numbers (unless there are discrepancies between our sequences and those in the database. Some sequences have been submitted multiple times and are redundant.
²GenBank Accession U55832 is actually a M. abscessus hsp65 instead M. chelonae ATCC 14472.
³GenBank Accessions AY299151 and AF547831 from M. flavescens ATCC 14474 do not match each other. They also do not match our sequence.
⁴AF547848 is from same strain but has 1 bp mismatch to all other three deposited sequences as well as our sequence.
⁵Our sequence matches AF547878 but is 1 bp different from AF434731.

### REFERENCES

1. Bercovier, H., O. Kafri, and S. Seta. 1986. Mycobacteria possess a surprisingly small number of ribosomal RNA genes in relation to the size of their genome. Biochem Biophys Res Commun 136:1136-41.
2. Billinger, M. E., K. N. Olivier, C. Viboud, R. M. de Oca, C. Steiner, S. M. Holland, and 1). R. Prevots. 2009. Nontuberculous mycobacteria-associated lung disease in hospitalized persons, United States, 1998-2005. Emerg Infect Dis 15:1562-9.
3. Blackwood, K. S., C. He, J. Gunton, C. Y. Turenne, J. Wolfe, and A. M. Kabani. 2000. Evaluation of recA sequences for identification of Mycobacterium species. J Clin Microbiol 38:2846-52.
4. Bodle, E. E., J. A. Cunningham, P. Della-Latta, N. W. Schluger, and L. Saiman. 2008. Epidemiology of nontuberculous mycobacteria in patients without HIV infection, New York City. Emerg Infect Dis 14:390-6.
5. Brosch, R., S. V. Gordon, T. Garnier, K. Eiglmeier, W. Frigui, P. Valenti, S. Dos Santos, S. Duthoy, C. Lacroix, C. Garcia-Pelayo, J. K. Inwald, P. Golby, J. N. Garcia, R. G. Hewinson, M. A. Behr, M. A. Quail, C. Churcher, B. G. Barrell, J. Parkhill, and S. T. Cole. 2007. Genome plasticity of BCG and impact on vaccine efficacy. Proc Natl Acad Sci USA 104:5596-601.
6. Brosch, R., S. V. Gordon, M. Marmiesse, P. Brodin, C. Buchrieser, K. Eiglmeier, T. Garnier, C. Gutierrez, G. Hewinson, K. Kremer, L. M. Parsons, A. S. Pym, S. Samper, D. van Soolingen, and S. T. Cole. 2002, A new evolutionary scenario for the Mycobacterium tuberculosis complex. Proc Natl Acad Sci USA 99:3684-9.
7. Brunello, F., M. Ligozzi, E. Cristelli, S. Bonora, E. Tortoli, and R. Fontana. 2001. Identification of 54 mycobacterial species by PCR-restriction fragment length polymorphism analysis of the hsp65 gene. J Clin Microbiol 39:2799-806.
8. Butler, W. R., and J. T. Crawford. 1999. Nontuberculous Mycobacteria Reported to the Public Health Laboratory Information System by State Public Health Laboratories United States, 1993-1996. Centers for Disease Control and Prevention.
9. Cerdeno-Tarraga, A. M., A. Efstratiou, L. G. Dover, M. T. Holden, M. Pallen, S.
D. Bentley, G. S. Besra, C. Churcher, K. D. James, A. De Zoysa, T. Chillingworth, A. Cronin, L. Dowd, T. Feltwell, N. Hamlin, S. Holroyd, K. Jagels, S. Moule, M. A. Quail, E. Rabbinowitsch, K. M. Rutherford, N. R. Thomson, L. Unwin, S. Whitehead, B. G. Barrell, and J. Parkhill. 2003. The complete genome sequence and analysis of Corynebacterium diphtheriae NCTC13129. Nucleic Acids Res 31:6516-23.
10. Cloeckaert, A., J. M. Verger, M. Grayon, and O. Grepinet. 1996. Polymorphism at the dnaK locus of Brucella species and identification of a Brucella melitensis species-specific marker. J Med Microbiol 45:200-5.
11. Cole, S. T., R. Brosch, J. Parkhill, T. Garnier, C. Churcher, D. Harris, S. V. Gordon, K. Eiglmeier, S. Gas, C. E. Barry, 3rd, F. Tekaia, K. Badcock, D. Basham, D. Brown, T. Chillingworth, R. Connor, R. Davies, K. Devlin, T. Feltwell, S. Gentles, N. Hamlin, S. Holroyd, T. Hornsby, K. Jagels, A. Krogh, J. McLean, S. Moule, L. Murphy, K. Oliver, J. Osborne, M. A. Quail, M. A. Rajandream, J. Rogers, S. Rutter, K. Seeger, J. Skelton, R. Squares, S. Squares, J. E. Sulston, K. Taylor, S. Whitehead, and B. G. Barrell. 1998. Deciphering the biology of Mycobacterium tuberculosis from the complete genome sequence. Nature 393:537-44.
12. Cole, S. T., K. Eiglmeier, J. Parkhill, K. D. James, N. R. Thomson, P. R. Wheeler, N. Honore, T. Garnier, C. Churcher, D. Harris, K. Mungall, D. Basham, D. Brown, T. Chillingworth, R. Connor, R. M. Davies, K. Devlin, S. Duthoy, T. Feltwell, A. Fraser, N. Hamlin, S. Holroyd, T. Hornsby, K. Jagels, C. Lacroix, J. Maclean, S. Moule, L. Murphy, K. Oliver, M. A. Quail, M. A. Rajandream, K. M. Rutherford, S. Rutter, K. Seeger, S. Simon, M. Simmonds, J. Skelton, R. Squares, S. Squares, K. Stevens, K. Taylor, S. Whitehead, J. R. Woodward, and B. G. Barrell. 2001. Massive gene decay in the leprosy bacillus. Nature 409:1007-11.
13. De Smet, K. A., I. N. Brown, M. Yates, and J. Ivanyi. 1995. Ribosomal internal transcribed spacer sequences are identical among Mycobacterium avium-intracellulare complex isolates from AIDS patients, but vary among isolates from elderly pulmonary disease patients. Microbiology 141 (Pt 10):2739-47.
14. Devulder, G., M. Perouse de Montclos, and J. P. Flandrois. 2005. A multigene approach to phylogenetic analysis using the genus Mycobacterium as a model. Int J Syst Evol Microbiol 55:293-302.
15. Ewing, B., and P. Green. 1998. Base-calling of automated sequencer traces using phred. II. Error probabilities. Genome Res 8:186-94.
16. Ewing, B., L. Hillier, M. C. Wendl, and P. Green. 1998. Base-calling of automated sequencer traces using phred. I. Accuracy assessment. Genome Res 8:175-85.
17. Fleischmann, R. D., D. Alland, J. A. Eisen, L. Carpenter, O. White, J. Peterson, R. DeBoy, R. Dodson, M. Gwinn, D. Haft, E. Hickey, J. F. Kolonay, W. C. Nelson, L. A. Umayam, M. Ermolaeva, S. L. Salzberg, A. Deleher, T. Utterback, J. Weidman, H. Khouri, J. Gill, A. Mikula, W. Bishai, W. R. Jacobs Jr, Jr., J. C. Venter, and C. M. Fraser. 2002. Whole-genome comparison of Mycobacterium tuberculosis clinical and laboratory strains. J Bacteriol 184:5479-90.
18. Frothingham, R., and K. H. Wilson. 1993. Sequence-based differentiation of strains in the Mycobacterium avium complex. J Bacteriol 175:2818-25.
19. Garnier, T., K. Eiglmeier, J. C. Camus, N. Medina, H. Mansoor, M. Pryor, S. Duthoy, S. Grondin, C. Lacroix, C. Monsempe, S. Simon, B. Harris, R. Atkin, J. Doggett, R. Mayes, L. Keating, P. R. Wheeler, J. Parkhill, B. G. Burrell, S. T. Cole, S. V. Gordon, and R. G. Hewinson. 2003. The complete genome sequence of Mycobacterium bovis. Proc Natl Acad Sci USA 100:7877-82.
20. Gingeras, T. R., G. Ghandour, E. Wang, A. Berno, P. M. Small, F. Drobniewski, D. Alland, E. Desmond, M. Holodniy, and J. Drenkow. 1998. Simultaneous genotyping and species identification using hybridization pattern recognition analysis of generic Mycobacterium DNA arrays. Genome Res 8:435-48.
21. Goh, K. S., M. Fabre, R. C. Huard, S. Schmid, C. Sola, and N. Rastogi. 2006. Study of the gyrB gene polymorphism as a tool to differentiate among Mycobacterium tuberculosis complex subspecies further underlines the older evolutionary age of ‘Mycobacterium canettii’. Mol Cell Probes 20:182-90.
22. Gordon, D., C. Abajian, and P. Green. 1998. Consed: a graphical tool for sequence finishing. Genome Res 8:195-202.
23. Hershkovitz, I., H. D. Donoghue, D. E. Minnikin, C. S. Besra, 0. Y. Lee, A. M. Gernaey, E. Galili, V. Eshed, C. L. Greenblatt, E. Lemma, G. K. Bar-Gal, and M. Spigelman. 2008. Detection and molecular characterization of 9,000-year-old Mycobacterium tuberculosis from a Neolithic settlement in the Eastern Mediterranean. PLoS One 3:e3426.
24. Huard, R. C., M. Fabre, P. de Haas, L. C. Lazzarini, D. van Soolingen, D.
Cousins, and J. L. Ho. 2006. Novel genetic polymorphisms that further delineate the phylogeny of the Mycobacterium tuberculosis complex. J Bacteriol 188:4271-87.
25. Ikeda, M., and S. Nakagawa. 2003. The Corynebacterium glutamicum genome: features and impacts on biotechnological processes. Appl Microbiol Biotechnol 62:99-109.
26. Ishikawa, J., A. Yamashita, Y. Mikami, Y. Hoshino, H. Kurita, K. Hotta, T. Shiba, and M. Hattori. 2004. The complete genomic sequence of Nocardia farcinica IFM 10152. Proc Natl Acad Sci USA 101:14925-30.
27. Kalinowski, J., B. Bathe, D. Bartels, N. Bischoff, M. Bott, A. Burkovski, N. Dusch, L. Eggeling, B. J. Eikmanns, L. Gaigalat, A. Goesmann, M. Hartmann, K. Huthmacher, R. Kramer, B. Linke, A. C. McHardy, F. Meyer, B. Mockel, W. Pfefferle, A. Puhier, D. A. Rey, C. Ruckert, O. Rupp, H. Sahm, V. F. Wendisch, I. Wiegrabe, and A. Tauch. 2003. The complete Corynebacterium glutamicum ATCC 13032 genome sequence and its impact on the production of L-aspartate-derived amino acids and vitamins. J Biotechnol 104:5-25.
28. Kasai, H., T. Ezaki, and S. Harayama. 2000. Differentiation of phylogenetically related slowly growing mycobacteria by their gyrB sequences. J Clin Microbiol 38:301-8.
29. Khan, K., J. Wang, and T. K. Marras. 2007. Nontuberculous mycobacterial sensitization in the United States: national trends over three decades. Am J Respir Crit Care Med 176:306-13.
30. Kim, B. J., S. H. Lee, M. A. Lyu, S. J. Kim, G. H. Bai, G. T. Chae, E. C. Kim, C. Y. Cha, and Y. H. Kook. 1999. Identification of mycobacterial species by comparative sequence analysis of the RNA polymerase gene (rpoB). J Clin Microbiol 37:1714-20.
31. Kim, H., S. H. Kim, T. S. Shim, M. N. Kim, G. H. Bai, Y. G. Park, S. H. Lee, G. T. Chae, C. Y. Cha, Y. H. Kook, and B. J. Kim. 2005. Differentiation of Mycobacterium species by analysis of the heat-shock protein 65 gene (hsp65). Int J Syst Evol Microbiol 55:1649-56.
32. Kirschner, P., and E. C. Bottger. 1998. Species identification of mycobacteria using rDNA sequencing. Methods Mol Biol 101:349-61.
33. Kumar, S., M. Nei, J. Dudley, and K. Tamura. 2008. MEGA: a biologist-centric software for evolutionary analysis of DNA and protein sequences. Brief Bioinform 9:299-306.
34. Larkin, M. A., G. Blackshields, N. P. Brown, R. Chenna, P. A. McGettigan, H. McWilliam, F. Valentin, I. M. Wallace, A. Wilm, R. Lopez, J. D. Thompson, T. J. Gibson, and D. G. Higgins. 2007. Clustal W and Clustal X version 2.0. Bioinformatics 23:2947-8.
35. Lazzarini, L. C., R. C. Huard, N. L. Boechat, H. M. Gomes, M. C. Oelemann, N. Kurepina, E. Shashkina, F. C. Mello, A. L. Gibson, M. J. Virginio, A. C. Marsico, W. R. Butler, B. N. Kreiswirth, P. N. Suffys, E. S. J. R. Lapa, and J. L. Ho. 2007. Discovery of a novel Mycobacterium tuberculosis lineage that is a major cause of tuberculosis in Rio de Janeiro, Brazil. J Clin Microbiol 45:3891-902.
36. Lee, H., H. J. Park, S. N. Cho, G. H. Bai, and S. J. Kim. 2000. Species identification of mycobacteria by PCR-restriction fragment length polymorphism of the rpoB gene. J Clin Microbiol 38:2966-71.
37. Li, L., J. P. Bannantine, Q. Zhang, A. Amonsin, B. J. May, D. Alt, N. Banerji, S. Kanjilal, and V. Kapur. 2005. The complete genome sequence of Mycobacterium avium subspecies paratuberculosis. Proc Natl Acad Sci USA 102:12344-9.
38. Martens, M., P. Dawyndt, R. Coopman, M. Gillis, P. De Vos, and A. Willems. 2008. Advantages of multilocus sequence analysis for taxonomic studies: a case study using 10 housekeeping genes in the genus Ensifer (including former Sinorhizobium). Int J Syst Evol Microbiol 58:200-14.
39. McLeod, M. P., R. L. Warren, W. W. Hsiao, N. Araki, M. Myhre, C. Fernandes, D. Miyazawa, W. Wong, A. L. Linguist, D. Wang, M. Dosanjh, H. Hara, A.
Petrescu, R. D. Morin, G. Yang, J. M. Stott, J. E. Schein, H. Shin, D. Smailus, A.
S. Siddiqui, M. A. Marra, S. J. Jones, R. Holt, F. S. Brinkman, K. Miyauchi, M. Fukuda, J. E. Davies, W. W. Mohn, and L. D. Eltis. 2006. The complete genome of Rhodococcus sp. RHA1 provides insights into a catabolic powerhouse. Proc Natl Acad Sci USA 103:15582-7.
40. Mignard, S., and J. P. Flandrois. 2007. Identification of Mycobacterium using the EF-Tu encoding (tuf) gene and the tmRNA encoding (ssrA) gene. J Mcd Microbiol 56:1033-41.
41. Mignard, S., and J. P. Flandrois. 2008. A seven-gene, multilocus, genus-wide approach to the phylogeny of mycobacteria using supertrees. Int J Syst Evol Microbiol 58:1432-41.
42. Miller, C. D., K. Hall, V. N. Liang, K. Nieman, D. Sorensen, B. Issa, A. J. Anderson, and R. C. Sims. 2004. Isolation and characterization of polycyclic aromatic hydrocarbon-degrading Mycobacterium isolates from soil. Microb Ecol 48:230-8.
43. Mostowy, S., J. Inwald, S. Gordon, C. Martin, R. Warren, K. Kremer, D. Cousins, and M. A. Behr. 2005. Revisiting the evolution of Mycobacterium Bovis. J Bacteriol 187:6386-95.
44. Nandasena, K. G., G. W. O'Hara, R. P. Tiwari, A. Willlems, and J. G. Howieson. 2007. Mesorhizobium ciceri biovar biserrulae, a novel biovar nodulating the pasture legume Biserrula pelecinus L. Int J Syst Evol Microbiol 57:1041-5.
45. Neumann, A. P., and T. G. Rehberger. 2009. MLST analysis reveals a highly conserved core genome among poultry isolates of Clostridium septicum. Anaerobe 15:99-106.
46. Niemann, S., D. Harmsen, S. Rusch-Gerdes, and E. Richter. 2000. Differentiation of clinical Mycobacterium tuberculosis complex isolates by gyrB DNA sequence polymorphism analysis. J Clin Microbiol 38:3231-4.
47. Ninet, B., M. Monod, S. Emler, J. Pawlowski, C. Metral, P. Rohner, R. Auckenthaler, and B. Hirschel. 1996. Two different 16S rRNA genes in a mycobacterial strain. J Clin Microbiol 34:2531-6.
48. Nishio, V., Y. Nakamura, Y. Kawarabayasi, Y. Usuda, E. Kimura, S. Sugimoto, K. Matsui, A. Yamagishi, H. Kikuchi, K. Ikeo, and T. Gojobori. 2003. Comparative complete genome sequence analysis of the amino acid replacements responsible for the thermostability of Corynebacterium efficiens. Genome Res 13:1572-9.
49. Picardeau, M., G. Prod'Hom, L. Raskine, M. P. LePennec, and V. Vincent. 1997. Genotypic characterization of five subspecies of Mycobacterium kansasii. J Clin Microbiol 35:25-32.
50. Rcischl, U., K. Feldmann, L. Naumann, B. J. Gaugler, B. Ninet, B. Hirschel, and S. Emler. 1998. 16S rRNA sequence diversity in Mycobacterium celatum strains caused by presence of two different copies of 16S rRNA gene. J Clin Microbiol 36:1761-4.
51. Ringuet, H., C. Akoua-Koffi, S. Honore, A. Varnerot, V. Vincent, P. Berche, J. L. Gaillard, and C. Pierre-Audigier. 1999. hsp65 sequencing for identification of rapidly growing mycobacteria. J Clin Microbiol 37:852-7.
52. Robbins, G., V. M. Tripathy, V. N. Misra, R. K. Mohanty, V. S. Shinde, K. M. Gray, and M. D. Schug. 2009. Ancient skeletal evidence for leprosy in India (2000 B.C.). PLoS One 4:e5669.
53. Roth, A., M. Fischer, M. E. Hamid, S. Michalke, W. Ludwig, and H. Mauch. 1998. Differentiation of phylogenetically related slowly growing mycobacteria based on 16S-23S rRNA gene internal transcribed spacer sequences. J Clin Microbiol 36:139-47.
54. Ryan, K. J., and J. C. Sherris. 1994. Sherris medical microbiology: an introduction to infectious diseases, 3rd ed. Appleton & Lange, Norwalk, Conn.
55. Soini, H., E. C. Bottger, and M. K. Viljanen. 1994. Identification of mycobacteria by PCR-based sequence determination of the 32-kilodalton protein gene. J Clin Microbiol 32:2944-7.
56. Soini, H., and M. K. Viljanen. 1997. Diversity of the 32-kilodalton protein gene may form a basis for species determination of potentially pathogenic mycobacterial species. J Clin Microbiol 35:769-73.
57. Stadthagen-Gomez, G., A. C. Helguera-Repetto, J. F. Cerna-Cortes, R. A. Goldstein, R. A. Cox, and J. A. Gonzalez-y-Merchand. 2008. The organization of two rRNA (rrn) operons of the slow-growing pathogen Mycobacterium celatum provides key insights into mycobacterial evolution. FEMS Microbiol Lett 280:102-12.
58. Stinear, T. P., T. Seemann, P. F. Harrison, G. A. Jenkin, J. K. Davies, P. D. Johnson, Z. Abdellah, C. Arrowsmith, T. Chillingworth, C. Churcher, K. Clarke, A. Cronin, P. Davis, I. Goodhead, N. Holroyd, K. Jagels, A. Lord, S. Moule, K. Mungall, H. Norbertczak, M. A. Quail, E. Rabbinowitsch, D. Walker, B. White, S. Whitehead, P. L. Small, R. Brosch, L. Ramakrishnan, M. A. Fischbach, J. Parkhill, and S. T. Cole. 2008. Insights from the complete genome sequence of Mycobacterium marinum on the evolution of Mycobacterium tuberculosis. Genome Res 18:729-41.
59. Stinear, T. P., T. Seemann, S. Pilot, W. Frigui, G. Reysset, T. Garnier, G. Meurice, D. Simon, C. Bouchier, L. Ma, M. Tichit, J. L. Porter, J. Ryan, P. D. Johnson, J. K. Davies, G. A. Jenkin, P. L. Small, L. M. Jones, F. Tekaia, F. Laval, M. Daffe, J. Parkhill, and S. T. Cole. 2007. Reductive evolution and niche adaptation inferred from the genome of Mycobacterium ulcerans, the causative agent of Buruli ulcer. Genome Res 17:192-200.
60. Takewaki, S., K. Okuzumi, H. Ishiko, K. Nakahara, A. Ohkubo, and R. Nagai.
1993. Genus-specific polymerase chain reaction for the mycobacterial dnaJ gene and species-specific oligonucleotide probes. J Clin Microbiol 31:446-50.
61. Tauch, A., O. Kaiser, T. Hain, A. Gocsmann, B. Weisshaar, A. Albersmeier, T. Bekel, N. Bischoff, 1. Brune, T. Chakraborty, J. Kalinowski, F. Meyer, O. Rupp, S. Schneiker, P. Viehoever, and A. Puhler. 2005. Complete genome sequence and analysis of the multiresistant nosocomial pathogen Corynebacterium jeikeium K411, a lipid-requiring bacterium of the human skin flora. J Bacteriol 187:4671-82.
62. Tauch, A., E. Trost, A. Tilker, U. Ludewig, S. Schneiker, A. Goesmann, W. Arnold, T. Bekel, K. Brinkrolf, I. Brune, S. Gotker, J. Kalinowski, P. B. Kamp, F. P. Lobo, P. Viehoever, B. Weisshaar, F. Soriano, M. Droge, and A. Puhler.
2008. The lifestyle of Corynebacterium urealyticum derived from its complete genome sequence established by pyrosequencing. J Biotcchnol 136:11-21.
63. Telenti, A., F. Marchesi, M. Balz, F. Bally, F. C. Bottger, and T. Bodmer. 1993. Rapid identification of mycobacteria to the species level by polymerase chain reaction and restriction enzyme analysis. J Clin Microbiol 31:175-8.
64. Teyssier, C., H. Marchandin, H. Jean-Pierre, A. Masnou, G. Dusart, and E. Jumas-Bilak, 2007. Ochrobactrum pseudintermedium sp. nov., a novel member of the family Brucellaceae, isolated from human clinical samples. Int J Syst Evol Microbiol 57:1007-13.
65. Turenne, C. Y., L. Tschetter, J. Wolfe, and A. Kabani. 2001. Necessity of quality-controlled 16S rRNA gene sequence databases: identifying nontuberculous Mycobacterium species. J Clin Microbiol 39:3637-48.
66. van der Giessen, J. W., R. M. Haring, and B. A. van der Zeijst. 1994. Comparison of the 23S ribosomal RNA genes and the spacer region between the 16S and 23S rRNA genes of the closely related Mycobacterium avium and Mycobacterium paratuberculosis and the fast-growing Mycobacterium phlei. Microbiology 140 (Pt 5):1103-8.
67. van Soolingen, D., T. Hoogenboezem, P. E. de Haas, P. W. Hermans, M. A. Koedam, K. S. Teppema, P. J. Brennan, G. S. Besra, F. Portaels, J. Top, L. M. Schouls, and J. D. van Embden. 1997. A novel pathogenic taxon of the Mycobacterium tuberculosis complex, Canetti: characterization of an exceptional isolate from Africa. Int J Syst Bacteriol 47:1236-45.
68. Wayne, L. G., and H. A. Sramek. 1992. Agents of newly recognized or infrequently encountered mycobacterial diseases. Clin Microbiol Rev 5:1-25.
69. Wei, G., W. Chen, J. P. Young, and C. Bontemps. 2009. A new clade of Mesorhizobium nodulating Alhagi sparsifolia. Syst Appl Microbiol 32:8-16.
70. Yamada-Noda, M., K. Ohkusu, H. Hata, M. M. Shah, P. H. Nhung, X. S. Sun, M. Hayashi, and T. Ezaki. 2007. Mycobacterium species identification—a new approach via dnaJ gene sequencing. Syst Appl Microbiol 30:453-62.
71. Young, J. C., V. R. Agashe, K. Siegers, and F. U. Hartl. 2004. Pathways of chaperone-mediated protein folding in the cytosol. Nat Rev Mol Cell Biol 5:781-91.
72. Young, J. M., D. C. Park, H. M. Shearman, and E. Fargier. 2008. A multilocus sequence analysis of the genus Xanthomonas. Syst Appl Microbiol 31:366-77.
73. Yukawa, H., C. A. Omumasaba, H. Nonaka, P. Kos, N. Okai, N. Suzuki, M. Suda, Y. Tsuge, J. Watanabe, Y. Ikeda, A. A. Vertes, and M. lnui. 2007. Comparative analysis of the Corynebacterium glutamicum group and complete genome sequence of strain R. Microbiology 153:1042-58.
74. Zelazny, A. M., L. B. Calhoun, L. Li, Y. R. Shea, and S. H. Fischer. 2005. Identification of Mycobacterium species by secA1 sequences. J Clin Microbiol 43:1051-8.
75. Zheng, H., L. Lu, B. Wang, S. Pu, X. Zhang, G. Zhu, W. Shi, L. Zhang, H. Wang, S. Wang, G. Zhao, and Y. Zhang. 2008. Genetic basis of virulence attenuation revealed by comparative genomic analysis of Mycobacterium tuberculosis strain H37Ra versus H37Rv. PLoS ONE 3:e2375.
76. Zolg, J. W., and S. Philippi-Schulz. 1994. The superoxide dismutase gene, a target for detection and identification of mycobacteria by PCR. J Clin Microbiol 32:2801-12.

1-10. (canceled)

11. An assay for detecting the hybridization or a probe or primer with a nucleic acid sequence in a sample comprising contacting a sample containing a target sequence with a probe or a primer comprising a nucleic acid sequence that hybridizes with one or more of the nucleic acid sequence selected from SEQ ID NOs: 1-46 under conditions that permit the hybridization of said probe or primer with said nucleic acid sequence(s) and detecting hybridization between said probe and said target sequence.

12. The assay according to claim 11, wherein said probe is selected from:
clpC1F1:
CGCTACCGCGGTGACTTCGA;
clpC1R1:
GGGCCGGCGAAGATGAACGA;
rpoBCF2:
CCTCGGAATCAACCTGTCCCGCAA;
rpoBCR2:
GTTCATCGAAGAAGTTGACGTC;
rpoBCF1:
GAGATGGAGTGCTGGGCCATGC;
rpoBCR1:
CCGAAGATCTTCTCGCAGAACAG;
dnaKF1:
CTGACCAAGGACAAGATGGC;
or
dnaKR1:
TCGATCAGCTTGGTCATCAC.

13. The assay according to claim 11, wherein said assay comprises the hybridization or a probe or primer with the clpC1, dnaK, and rpoBC loci.

14. The assay according to claim 12, wherein said assay comprises the hybridization or a probe or primer with the clpC1, dnaK, and rpoBC loci.

15. A primer pair selected from:
a)
clpC1F1:
CGCTACCGCGGTGACTTCGA
and
clpC1R1:
GGGCCGGCGAAGATGAACGA;
b)
rpoBCF2:
CCTCGGAATCAACCTGTCCCGCAA
and
rpoBCR2:
GTTCATCGAAGAAGTTGACGTC;
c)
rpoBCF1:
GAGATGGAGTGCTGGGCCATGC
and
rpoBCR1:
CCGAAGATCTTCTCGCAGAACAG;
or
d)
dnaKF1:
CTGACCAAGGACAAGATGGC;
and
dnaKR1:
TCGATCAGCTTGGTCATCAC.

16. A nucleic acid probe or primer that hybridizes to a nucleic acid sequence selected from any one of SEQ ID NOs: 1-46.

17. A composition comprising at least one primer pair according to claim 15.

18. A composition comprising at least one nucleic acid probe or primer according to claim 16.

19. The composition according to claim 18, wherein said at least one probe or primer is selected from:
clpC1F1:
CGCTACCGCGGTGACTTCGA;
clpC1R1:
GGGCCGGCGAAGATGAACGA;
rpoBCF2:
CCTCGGAATCAACCTGTCCCGCAA;
rpoBCR2:
GTTCATCGAAGAAGTTGACGTC;
rpoBCF1:
GAGATGGAGTGCTGGGCCATGC;
rpoBCR1:
CCGAAGATCTTCTCGCAGAACAG;
dnaKF1:
CTGACCAAGGACAAGATGGC;
or
dnaKR1:
TCGATCAGCTTGGTCATCAC.

20. A nucleic acid array comprising a solid substrate and at least one probe or primer according to claim 16.

21. The nucleic acid array according to claim 20, wherein said at least one probe or primer is selected from:
clpC1F1:
CGCTACCGCGGTGACTTCGA;
clpC1R1:
GGGCCGGCGAAGATGAACGA;
rpoBCF2:
CCTCGGAATCAACCTGTCCCGCAA;
rpoBCR2:
GTTCATCGAAGAAGTTGACGTC;
rpoBCF1:
GAGATGGAGTGCTGGGCCATGC;
rpoBCR1:
CCGAAGATCTTCTCGCAGAACAG;
dnaKF1:
CTGACCAAGGACAAGATGGC;
or
dnaKR1:
TCGATCAGCTTGGTCATCAC.

22. A nucleic acid array comprising a solid substrate and a nucleic acid consisting essentially of one or more of SEQ ID NOs: 1-46.


SEQUENCE LISTING (46 sequences)

SEQ ID NO: 1 - DNA, 832 bp, Mycobacterium tuberculosis H37Rv
ggctacatgtacatcatgaagctgcaccacctggtggacgacaagatccacgcccgctccaccgggccgtactcgatgatcacccagcagccgctgggcggtaaggcgcagttcggtggccagcggttcggggagatggagtgctgggccatgcaggcctacggtgctgcctacaccctgcaggagctgttgaccatcaagtccgatgacaccgtcggccgcgtcaaggtgtacgaggcgatcgtcaagggtgagaacatcccggagccgggcatccccgagtcgttcaaggtgctgctcaaagaactgcagtcgctgtgcctcaacgtcgaggtgctatcgagtgacggtgcggcgatcgaactgcgcgaaggtgaggacgaggacctggagcgggccgcggccaacctgggaatcaatctgtcccgcaacgaatccgcaagtgtcgaggatcttgcgtaaagctgtcgcaaaattactaaacccgttaggggaaagggagttacgtgctcgacgtcaacttcttcgatgaactccgcatcggtcttgctaccgcggaggacatcaggcaatggtcctatggcgaggtcaaaaagccggagacgatcaactaccgcacgcttaagccggagaaggacggcctgttctgcgagaagatcttcgggccgactcgcgactgggaatgctactgcggcaagtacaagcgggtgcgcttcaagggcatcatctgcgagcgctgcggcgtcgaggtgacccgcgccaaggtgcgtcgtgagcggatgggccacatcgagcttgccgcgcccgtcacccacatctggtacttcaaggg

SEQ ID NO: 2 - DNA, 201 bp, Mycobacterium tuberculosis H37Rv
cgcgattcgatgttccaggcgatacttccgctgcgcggcaagatcatcaatgtggagaaagcgcgcatcgaccgggtgctaaagaacaccgaagttcaggcgatcatcacggcgctgggcaccgggatccacgacgagttcgatatcggcaagctgcgctaccacaagatcgtgctgatggccgacgccgatgttgacggc

SEQ ID NO: 3 - DNA, 1334 bp, Mycobacterium tuberculosis H37Rv
gtcggccagcccgccaagaaccaggcagtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccga

SEQ ID NO: 4 - DNA, 329 bp, Mycobacterium tuberculosis H37Rv
aggttggacgtcaggatcaagatggtgttgcggaagtcgaccgtgcggccgtgcccgtcggtgagccggccctcgtcgaggacctgcagcagcacgtcgaacacgtccgggtgcgccttctcgatctcgtcgaacagcaccaccgtgtagggacgccggcgcaccgcctcggtcagctgaccgcccgcctcgtatcccacatagccgggcggggcgccgatcaaccgagccacggtgtgcttctcgccgtactcgctcatgtcgatgcggaccatcgcccgctcgtcgtcgaacaggaagtcggccagcgccttggccagctcggtctt

SEQ ID NO: 5 - DNA, 508 bp, Mycobacterium tuberculosis H37Rv
ccggtaagccgctgctgatcatcgccgaggacgtcgagggcgaggcgctgtccaccctggtcgtcaacaagatccgcggcaccttcaagtcggtggcggtcaaggctcccggcttcggcgaccgccgcaaggcgatgctgcaggatatggccattctcaccggtggtcaggtgatcagcgaagaggtcggcctgacgctggagaacgccgacctgtcgctgctaggcaaggcccgcaaggtcgtggtcaccaaggacgagaccaccatcgtcgagggcgccggtgacaccgacgccatcgccggacgagtggcccagatccgccaggagatcgagaacagcgactccgactacgaccgtgagaagctgcaggagcggctggccaagctggccggtggtgtcgcggtgatcaaggccggtgccgccaccgaggtcgaactcaaggagcgcaagcaccgcatcgaggatgcggttcgcaatgccaaggccgccgtcgaggagggcatcgt

SEQ ID NO: 6 - DNA, 2963 bp, Mycobacterium tuberculosis H37Rv
cagacggtgttcatgggtgacttcccgatgatgaccgagaagggcacgttcatcatcaacgggaccgagcgtgtggtggtcagccagctggtgcggtcgcccggggtgtacttcgacgagaccattgacaagtccaccgacaagacgctgcacagcgtcaaggtgatcccgagccgcggcgcgtggctcgagtttgacgtcgacaagcgcgacaccgtcggcgtgcgcatcgaccgcaaacgccggcaaccggtcaccgtgctgctcaaggcgctgggctggaccagcgagcagattgtcgagcggttcgggttctccgagatcatgcgatcgacgctggagaaggacaacaccgtcggcaccgacgaggcgctgttggacatctaccgcaagctgcgtccgggcgagcccccgaccaaagagtcagcgcagacgctgttggaaaacttgttcttcaaggagaagcgctacgacctggcccgcgtcggtcgctataaggtcaacaagaagctcgggctgcatgtcggcgagcccatcacgtcgtcgacgctgaccgaagaagacgtcgtggccaccatcgaatatctggtccgcttgcacgagggtcagaccacgatgaccgttccgggcggcgtcgaggtgccggtggaaaccgacgacatcgaccacttcggcaaccgccgcctgcgtacggtcggcgagctgatccaaaaccagatccgggtcggcatgtcgcggatggagcgggtggtccgggagcggatgaccacccaggacgtggaggcgatcacaccgcagacgttgatcaacatccggccggtggtcgccgcgatcaaggagttcttcggcaccagccagctgagccaattcatggaccagaacaacccgctgtcggggttgacccacaagcgccgactgtcggcgctggggcccggcggtctgtcacgtgagcgtgccgggctggaggtccgcgacgtgcacccgtcgcactacggccggatgtgcccgatcgaaacccctgaggggcccaacatcggtctgatcggctcgctgtcggtgtacgcgcgggtcaacccgttcgggttcatcgaaacgccgtaccgcaaggtggtcgacggcgtggttagcgacgagatcgtgtacctgaccgccgacgaggaggaccgccacgtggtggcacaggccaattcgccgatcgatgcggacggtcgcttcgtcgagccgcgcgtgctggtccgccgcaaggcgggcgaggtggagtacgtgccctcgtctgaggtggactacatggacgtctcgccccgccagatggtgtcggtggccaccgcgatgattcccttcctggagcacgacgacgccaaccgtgccctcatgggggcaaacatgcagcgccaggcggtgccgctggtccgtagcgaggccccgctggtgggcaccgggatggagctgcgcgcggcgatcgacgccggcgacgtcgtcgtcgccgaagaaagcggcgtcatcgaggaggtgtcggccgactacatcactgtgatgcacgacaacggcacccggcgtacctaccggatgcgcaagtttgcccggtccaaccacggcacttgcgccaaccagtgccccatcgtggacgcgggcgaccgagtcgaggccggtcaggtgatcgccgacggtccctgtactgacgacggcgagatggcgctgggcaagaacctgctggtggccatcatgccgtgggagggccacaactacgaggacgcgatcatcctgtccaaccgcctggtcgaagaggacgtgctcacctcgatccacatcgaggagcatgagatcgatgctcgcgacaccaagctgggtgcggaggagatcacccgcgacatcccgaacatctccgacgaggtgctcgccgacctggatgagcggggcatcgtgcgcatcggtgccgaggttcgcgacggggacatcctggtcggcaaggtcaccccgaagggtgagaccgagctgacgccggaggagcggctgctgcgtgccatcttcggtgagaaggcccgcgaggtgcgcgacacttcgctgaaggtgccgcacggcgaatccggcaaggtgatcggcattcgggtgttttcccgcgaggacgaggacgagttgccggccggtgtcaacgagctggtgcgtgtgtatgtggctcagaaacgcaagatctccgacggtgacaagctggccggccggcacggcaacaagggcgtgatcggcaagatcctgccggttgaggacatgccgttccttgccgacggcaccccggtggacattattttgaacacccacggcgtgccgcgacggatgaacatcggccagattttggagacccacctgggttggtgtgcccacagcggctggaaggtcgacgccgccaagggggttccggactgggccgccaggctgcccgacgaactgctcgaggcgcagccgaacgccattgtgtcgacgccggtgttcgacggcgcccaggaggccgagctgcagggcctgttgtcgtgcacgctgcccaaccgcgacggtgacgtgctggtcgacgccgacggcaaggccatgctcttcgacgggcgcagcggcgagccgttcccgtacccggtcacggttggctacatgtacatcatgaagctgcaccacctggtggacgacaagatccacgcccgctccaccgggccgtactcgatgatcacccagcagccgctgggcggtaaggcgcagttcggtggccagcggttcggggagatggagtgctgggccatgcaggcctacggtgctgcctacaccctgcaggagctgttgaccatcaagtccgatgacaccgtcggccgcgtcaaggtgtacgaggcgatcgtcaagggtgagaacatcccggagccgggcatccccgagtcgttcaaggtgctgctcaaagaactgcagtcgctgtgcctcaacgtcgaggt

SEQ ID NO: 7 - DNA, 1768 bp, Mycobacterium tuberculosis H37Rv
acgtgctcgacgtcaacttcttcgatgaactccgcatcggtcttgctaccgcggaggacatcaggcaatggtcctatggcgaggtcaaaaagccggagacgatcaactaccgcacgcttaagccggagaaggacggcctgttctgcgagaagatcttcgggccgactcgcgactgggaatgctactgcggcaagtacaagcgggtgcgcttcaagggcatcatctgcgagcgctgcggcgtcgaggtgacccgcgccaaggtgcgtcgtgagcggatgggccacatcgagcttgccgcgcccgtcacccacatctggtacttcaagggtgtgccctcgcggctggggtatctgctggacctggccccgaaggacctggagaagatcatctacttcgctgcctacgtgatcacctcggtcgacgaggagatgcgccacaatgagctctccacgctcgaggccgaaatggcggtggagcgcaaggccgtcgaagaccagcgcgacggcgaactagaggcccgggcgcaaaagctggaggccgacctggccgagctggaggccgagggcgccaaggccgatgcgcggcgcaaggttcgcgacggcggcgagcgcgagatgcgccagatccgtgaccgcgcgcagcgtgagctggaccggttggaggacatctggagcactttcaccaagctggcgcccaagcagctgatcgtcgacgaaaacctctaccgcgaactcgtcgaccgctacggcgagtacttcaccggtgccatgggcgcggagtcgatccagaagctgatcgagaacttcgacatcgacgccgaagccgagtcgctgcgggatgtcatccgaaacggcaaggggcagaagaagcttcgcgccctcaagcggctgaaggtggttgcggcgttccaacagtcgggcaactcgccgatgggcatggtgctcgacgccgtcccggtgatcccgccggagctgcgcccgatggtgcagctcgacggcggccggttcgccacgtccgacttgaacgacctgtaccgcagggtgatcaaccgcaacaaccggctgaaaaggctgatcgatctgggtgcgccggaaatcatcgtcaacaacgagaagcggatgctgcaggaatccgtggacgcgctgttcgacaatggccgccgcggccggcccgtcaccgggccgggcaaccgtccgctcaagtcgctttccgatctgctcaagggcaagcagggccggttccggcagaacctgctcggcaagcgtgtcgactactcgggccggtcggtcatcgtggtcggcccgcagctcaagctgcaccagtgcggtctgcccaagctgatggcgctggagctgttcaagccgttcgtgatgaagcggctggtggacctcaaccatgcgcagaacatcaagagcgccaagcgcatggtggagcgccagcgcccccaagtgtgggatgtgctcgaagaggtcatcgccgagcacccggtgttgctgaaccgcgcacccaccctgcaccggttgggtatccaggccttcgagccaatgctggtggaaggcaaggccattcagctgcacccgttggtgtgtgaggcgttcaatgccgacttcgacggtgaccagatggccgtgcacctgcctttgagcgccgaagcgcaggccgaggctcgcattttgatgttgtcctccaacaacatcctgtcgccggcatctgggcgtccgttggccatgccgcggctggacatggtgaccgggctgtactacctgac

SEQ ID NO: 8 - DNA, 409 bp, Mycobacterium tuberculosis H37Rv
ggtccgcaacttcggcatcatggcgcacatcgatgccggcaagaccacaaccaccgagcgcatcctgtactacaccggtatcaactacaagattggtgaggtgcacgacggcgcagccaccatggactggatggaacaggaacaggagcgcggcatcaccatcacctctgcggccacgaccacgttctggaaagacaaccagctcaatatcatcgacacgccagggcatgtggatttcaccgtcgaggtggagcgcaatctgcgcgtgctcgacggcgcggtcgcggttttcgacggcaaagagggtgtcgaaccgcagtccgaacaggtgtggcggcaggccgacaaatacgatgtcccccgaatctgcttcgtcaacaagatggacaagatcggtgcggacttctac

SEQ ID NO: 9 - DNA, 1201 bp, Mycobacterium tuberculosis H37Rv
agtccaggaggacacaaaagtggcgaaggcgaagttccagcggaccaagccccacgtcaacatcgggaccatcggtcacgttgaccacggcaagaccaccctgaccgcggctatcaccaaggtcctgcacgacaaattccccgatctgaacgagacgaaggcattcgaccagatcgacaacgcccccgaggagcgtcagcgcggtatcaccatcaacatcgcgcacgtggagtaccagaccgacaagcggcactacgcacacgtcgacgcccctggccacgccgactacatcaagaacatgatcaccggcgccgcgcagatggacggtgcgatcctggtggtcgccgccaccgacggcccgatgccccagacccgcgagcacgttctgctggcgcgtcaagtgggtgtgccctacatcctggtagcgctgaacaaggccgacgcagtggacgacgaggagctgctcgaactcgtcgagatggaggtccgcgagctgctggctgcccaggaattcgacgaggacgccccggttgtgcgggtctcggcgctcaaggcgctcgagggtgacgcgaagtgggttgcctctgtcgaggaactgatgaacgcggtcgacgagtcgattccggacccggtccgcgagaccgacaagccgttcctgatgccggtcgaggacgtcttcaccattaccggccgcggaaccgtggtcaccggacgtgtggagcgcggcgtgatcaacgtgaacgaggaagttgagatcgtcggcattcgcccatcgaccaccaagaccaccgtcaccggtgtggagatgttccgcaagctgctcgaccagggccaggcgggcgacaacgttggtttgctgctgcggggcgtcaagcgcgaggacgtcgagcgtggccaggttgtcaccaagcccggcaccaccacgccgcacaccgagttcgaaggccaggtctacatcctgtccaaggacgagggcggccggcacacgccgttcttcaacaactaccgtccgcagttctacttccgcaccaccgacgtgaccggtgtggtgacactgccggagggcaccgagatggtgatgcccggtgacaacaccaacatctcggtgaagttgatccagcccgtcgccatggacgaaggtctgcgtttcgcgatccgcgagggtggccgcaccgtgggcgccggccgggtcaccaagatc

SEQ ID NO: 10 - DNA, 296 bp, Mycobacterium tuberculosis H37Rv
agcgtggcgggacagaagatccgcatcaggctgaaggcctacgaccatgaggccattgacgcttcggcgcgcaagatcgtcgaaaccgtcgtccgcaccggtgccagcgtcgtagggccggtgccgctaccgactgagaagaacgtgtattgcgtcatccgctcaccgcataagtacaaggactcgcgggagcacttcgagatgcgcacacacaagcggttgatcgacatcatcgatcccacgccgaagaccgttgacgcgctcatgcgcatcgaccttccggccagcgtcgacgt

SEQ ID NO: 11 - DNA, 343 bp, Mycobacterium tuberculosis H37Rv
aggacaccgcgctggtattcggacagatggacgagccgccgggcacccgtatgcgtgttgcgctgtctgcgctgacgatggcggagtggttccgtgacgagcagggtcaagacgtattgctgttcatcgacaacatcttccggttcacccaggctgggtcggaagtgtcgacgcttctcggccggatgccgtcggccgtgggataccagcccacgctggccgacgagatgggcgagctgcaggagcgcatcacctcgacgcggggacgctcgatcacgtcgatgcaagccgtctacgtgcccgccgacgactacaccgacccagcgccggcgaccacgttcgc

SEQ ID NO: 12 - DNA, 1449 bp, Mycobacterium tuberculosis H37Rv
gagtggcgaacgggtgagtaacacgtgggtgatctgccctgcacttcgggataagcctgggaaactgggtctaataccggataggaccacgggatgcatgtcttgtggtggaaagcgctttagcggtgtgggatgagcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcaccatcgacgaaggtccgggttctctcggattgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcacgtaatggtggggactcgtgagagactgccggggtcaactcggaggaaggtggggatgacgtcaagtcatcatgccccttatgtccagggcttcacacatgctacaatggccggtacaaagggctgcgatgccgcgaggttaagcgaatccttaaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaaccctcgggagggagctgtcgaaggtgggatcggcgattgggacgaagtcgtaacaaggtagccgtaccggaaggtgcggctggatcacctcctttctaaggag

SEQ ID NO: 13 - DNA, 2208 bp, Mycobacterium tuberculosis H37Rv
tggacccgaagcggagtgatctacccatggccagggtgaagcgcgggtaagaccgcgtggaggcccgaacccacttaggttgaagactgaggggatgagctgtgggtaggggtgaaaggccaatcaaactccgtgatagctggttctccccgaaatgcatttaggtgcagcgttgcgtggttcaccgcggaggtagagctactggatggccgatgggccctactaggttactgacgtcagccaaactccgaatgccgtggtgtaaagcgtggcagtgagacggcgggggataagctccgtacgtcgaaagggaaacagcccagatcgccggctaaggcccccaagcgtgtgctaagtgggaaaggatgtgcagtcgcaaagacaaccaggaggttggcttagaagcagccacccttgaaagagtgcgtaatagctcactggtcaagtgattgtgcgccgataatgtagcggggctcaagcacaccgccgaagccgcggcacatccaccttgtggtgggtgtgggtaggggagcgtccctcattcagcgaagccaccgggtgaccggtggtggagggtgggggagtgagaatgcaggcatgagtagcgacaaggcaagtgagaaccttgcccgccgaaagaccaagggttcctgggccaggccagtccgcccagggtgagtcgggacctaaggcgaggccgacaggcgtagtcgatggacaacgggttgatattcccgtacccgtgtgtgggcgcccgtgacgaatcagcggtactaaccacccaaaaccggatcgatcactccccttcgggggtgtggagttctggggctgcgtgggaacttcgctggtagtagtcaagcgaaggggtgacgcaggaaggtagccgtaccagtcagtggtaacactggggcaagccggtagggagagcgataggcaaatccgtcgctcactaatcctgagaggtgacgcatagccggttgaggcgaattcggtgatcctctgctgccaagaaaagcctctagcgagcacacacacggcccgtaccccaaaccgacacaggtggtcaggtagagcataccaaggcgtacgagataactatggttaaggaactcggcaaaatgcccccgtaacttcgggagaagggggaccggaatatcgtgaacacccttgcggtgggagcgggatccggtcgcagaaaccagtgaggagcgactgtttactaaaaacacaggtccgtgcgaagtcgcaagacgatgtatacggactgacgcctgcccggtgctggaaggttaagaggacccgttaacccgcaagggtgaagcggagaatttaagccccagtaaacggcggtggtaactataaccatcctaaggtagcgaaattccttgtcgggtaagttccgacctgcacgaatggcgtaacgacttctcaactgtctcaaccatagactcggcgaaattgcactacgagtaaagatgctcgttacgcgcggcaggacgaaaagaccccgggaccttcactacaacttggtattgatgttcggtacggtttgtgtaggataggtgggagactgtgaaacctcgacgccagttggggcggagtcgttgttgaaataccactctgatcgtattgggcatctaacctcgaaccctgaatcgggtttagggacagtgcctggcgggtagtttaactggggcggttgcctcctaaaatgtaacggaggcgcccaaaggttccctcaacctggacggcaatcaggtggcgagtgtaaatgcacaagggagcttgactgcgagacttacaagtcaagcagggacgaaagtcgggattagtgatccggcacccccgagtggaaggggtgtcgctcaacggataaaaggtaccccggggataacaggctgatcttccccaagagtccatatcgacgggatggtttggcacctcgatgtcggctcgtcgcatcctggggctggagcaggtcccaagggttgggctgttcgcccattaaagcggcacgcgagctgggtttagaacgtcgtgagacagttcggtctctatccgccgcgcgcgtcagaaacttgaggaaacctgtccctagtacgagaggaccgggacggacgaacctctggtgcaccagttgtcccgccaggggcaccgctggatagccacgttcggtcaggataaccgctgaaagcatctaagcgggaa

SEQ ID NO: 14 - DNA, 1130 bp, Mycobacterium tuberculosis H37Rv
atcaagtacttcaacgatggcgacatcgtcgaaggcaccatcgtcaaagtggaccgggacgaggtgctcctcgacatcggctacaagaccgaaggcgtgatccccgcccgcgaactgtccatcaagcacgacgtcgaccccaacgaggtcgtttccgtcggtgacgaggtcgaagccctggtgctcaccaaggaggacaaagagggccggctcatcctctccaagaaacgcgcgcagtacgagcgtgcctggggcaccatcgaggcgctcaaggagaaggacgaggccgtcaagggcacggtcatcgaggtcgtcaagggtggcctgatcctcgacatcgggctgcgcggtttcctgcccgcctcgctggtggagatgcgccgggtgcgcgacctgcagccctacatcggcaaggagatcgaggccaagatcatcgagctggacaagaaccgcaacaacgtggtgctgtcccgtcgcgcctggctggagcagacccagtccgaggtgcgcagcgagttcctgaataacttgcaaaaaggcaccatccgaaagggtgtcgtgtcctcgatcgtcaacttcggcgcgttcgtcgatctcggcggtgtggacggtctggtgcatgtctccgagctatcgtggaagcacatcgaccacccgtccgaggtggtccaggttggtgacgaggtcaccgtcgaggtgctcgacgtcgacatggaccgtgagcgggtttcgttgtcactcaaggcgactcaggaagacccgtggcggcacttcgcccgcactcacgcgatcgggcagatcgtgccgggcaaggtcaccaagttggttccgttcggtgcattcgtccgcgtcgaggagggtatcgagggcctggtgcacatctccgagctggccgagcgtcacgtcgaggtgcccgatcaggtggttgccgtcggcgacgacgcgatggtcaaggtcatcgacatcgacctggagcgccgtcggatctcgttgtcgctcaagcaagccaatgaggactacaccgaggagttcgacccggcgaagtacggcatggccgacagttacgacgagcagggcaactacatcttccccgagggcttcgatgccgaaaccaacgaatggcttgaggg

SEQ ID NO: 15 - DNA, 416 bp, Mycobacterium tuberculosis H37Rv
aaccatctgctggaagccaacctgcgcctggtggtttcgctagccaagcgctacaccggccggggcatggcgtttctcgacctgatccaggaaggcaacctggggctgatccgcgcggtggagaagttcgactacaccaaggggtacaagttctccacctacgctacgtggtggattcgccaggccatcacccgcgccatggccgaccaggcccgcaccatccgcatcccggtgcacatggtcgaggtgatcaacaagctgggccgcattcaacgcgagctgctgcaggacctgggccgcgagcccacgcccgaggagctggccaaagagatggacatcaccccggagaaggtgctggaaatccagcaatacgcccgcgagccgatctcgttggaccagaccatcggcgacgaggg

SEQ ID NO: 16 - DNA, 1543 bp, Mycobacterium tuberculosis H37Rv
cgcagccggatgtagtacagcgtcttgatccccttgcgccaggcgtaaatctgcgccttgttcacgtcgcgggtggtggcggtgtctttgaagaacaacgtcagcgaaagcccttgatccacatgctgggtggccgccgcgtaggtgtcgatgatcttctcgtaaccgatctcgtaggcgtcttcgtagtactccaggttgtcgttggtcatatacggcgccgggtagtagacccgcccgatcttgccttccttgcggatctcgaccttcgacacgatcgggtgaatcgacgacgtcgaatggttgatgtaggaaatcgacccggtcggcggcaccgcctgcaggttctggttgtagatgccgtgcgcttgcaccgactccttgagccgacgccagtcgtcctgcgttgggatgcggatgccggcgtcggcgaacagctggcgtaccttctgggtcttcggctcccaaatctggtcggtgtacttgtcgaagaattccccggacgcgtacttggaccgctcgaaacccttgaagtgcgtgccgcgttcgatcgcgatgcggttggatgcccgcaacgcgtgatacagcaccgtatagaagtagatgttggtgaagtcgatgccttcgtcggatccgtagaagatgcgttcccgggccaggtagccgtgcaggttcatctgtcctagcccgatcgcgtgggagtcgttgttgccctgctcgattgagggcaccgacttgatatgggtttggtcgctcaccgcggtcaacgcgcggatcgccacctcgatcgtctgcgcgaagtccggcgagtccatcgtcttggcgatgttcagcgaccccaggttgcacgaaatgtctttgcccactttggcatacgacaagtcctcgttgaacaatgacggcgtagacacttgcaggatctccgagcacaggttgctgtgcgtgatcttgccatcaattggattagcgcgattgacggtgtcttcgaacatgatataggggtagccggactcgaactgcagctcggccagcgtctggaagaactcccgtgccttgatcttggtcttgcggatgcgcgcgtcatcgaccatttcgtagtacttctcggtgaccgagatgtcagcgaacggcacaccgtagacccgctcgacatcgtagggcgagaacaggtacatgtcatcgttgcgcttggccaactcgaaggtgatgtcggggatcaccacccccagactcagcgtcttgatccggatcttctcgtcggcgttctcacgcttggtgtccaggaatcggtagatgtcggggtgatgggcgtgcaggtacaccgcgccggcaccttgacgagcgcccagctggttggcgtaggagaacgcatcctccagcaacttcatgatggggatgacgcccgaggactggttctcgatgttcttgatcggcgcgccgtgctcgcgaatgttggtcagcagcaacgccactcccccgccacgcttggatagctgcagcgcggagttgatcgaccgtccgatcgactccatgttgtcttcga

SEQ ID NO: 17 - DNA, 1512 bp, Mycobacterium tuberculosis H37Rv
cttagagatgtcggaggtgcccagattggacgtaaagatcagcacggtgttcttgaagtccaccgtgcggccctgcccgtcggtgagccggccatcctcgagcacctgcagcaggctgttgtagatctcctgatgcgccttctcgatctcgtcgaacagcaccaccgagaacggcttgcgccgcaccttctcggtgagttggccgccctcctcgtagccgacgtatccgggcggcgcgccgaatagccgcgacgcggtgaaccggtcgtggaattcacccatgtcaatctgaataagcgcgtcgtcgtcaccgaacaagaagttggccagcgccttggacagttcggtcttaccgacaccggacgggccggcgaagatgaacgagcccgacgggcgcttggggtctttcagcccggcccgggtacgccggatggccttggaaacggccttgacggcgtcctcttgcccgatgatccgcttgtgcagctcttcttccatccgcaacagccgggtggtctcggcctcggtgagcttgaacaccgggataccggtccagttgcccagcacctcggcgatctgctcgtcgtcgacctccgcgaccacgtcaagatcgcctgaacgccactgcttttcgcgctcagcacgctgtgcgaccagtgtcttctcccggtcgcgcaggctggcggccttctcgaagtcctgggcgtcgatagccgattccttctcccgacgagcctcggcgatcttctcatcgaactcgcgtaggtctggcggtgcggtcatgcgacgaatccgcatccgagcacccgcctcgtcgatcaggtcgatcgccttgtcgggcaggaaccggtcgttgatgtagcggtcggccagggtcgcggcggccaccatcgccgcatcggtgatcgacacccggtggtgcgcctcgtaccggtcccgcaggcccttgaggatctcgatggtgtgctccaccgtcggctcacccacctgcaccggctggaagcggcgctccagcgcggcgtccttctcgatgtacttgcggtattcgtcgagcgtggtggcgccgatcgtttgcagttcaccgcgagcgagcttcggtttcaggatcgaggcggcgtcgatcgcgccctcggcggctccagcaccgaccaaggtgtgcagctcgtcgataaacaggatgatgtcaccgcgggtgttgatctccttgagcaccttcttgaggcgttcctcgaagtcaccgcggtagcggctacccgccaccagcgatcccagatccagcgtgtagagctgcttgtccttgagcgtctcgggcacctcgccgtgcacgatggcctgcgccagtccttcgacgaccgcggtcttgccgacgccgggctcgccgatcagcaccgggttgttcttggtgcgccgagagagcacctgcatgacccgctcgatttccttctcgcggccgatgaccgggtccagtttgccttccatcgccgccgccgtgaggttgcggccgaactggtc

SEQ ID NO: 18 - DNA, 338 bp, Mycobacterium tuberculosis H37Rv
gggatctggcggtcgaagcggcccggccgcaacagcgccgggtccaggatgtcgggccggttggtggccgcgatcaggatgacgccggcgcgatcgccaaaaccgtccatttcgactagcaactggttgagggtctgctcacgctcgtcgtgaccgccgcccagcccggcgcctctttgtcggccgacggcgtcgatctcgtcgacgaagatgatgcacgggctgttctgcttggcctgctcgaacaggtctctgacacgggatgcgccgacgccgacgaacatttcgacgaagtcggagccggagatggtgaagaacggcactccggcttcgccggc

SEQ ID NO: 19 - DNA, 201 bp, Mycobacterium tuberculosis H37Rv; identical to SEQ ID NO: 2

SEQ ID NO: 20 - DNA, 588 bp, Mycobacterium tuberculosis H37Rv
gtcggccagcccgccaagaaccaggcagtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctg

SEQ ID NO: 21 - DNA, 566 bp, Mycobacterium tuberculosis H37Rv
gagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccga

SEQ ID NO: 22 - DNA, 329 bp, Mycobacterium tuberculosis H37Rv; identical to SEQ ID NO: 4

SEQ ID NO: 23 - DNA, 508 bp, Mycobacterium tuberculosis H37Rv; identical to SEQ ID NO: 5

SEQ ID NO: 24 - DNA, 327 bp, Mycobacterium tuberculosis H37Rv
cagacggtgttcatgggtgacttcccgatgatgaccgagaagggcacgttcatcatcaacgggaccgagcgtgtggtggtcagccagctggtgcggtcgcccggggtgtacttcgacgagaccattgacaagtccaccgacaagacgctgcacagcgtcaaggtgatcccgagccgcggcgcgtggctcgagtttgacgtcgacaagcgcgacaccgtcggcgtgcgcatcgaccgcaaacgccggcaaccggtcaccgtgctgctcaaggcgctgggctggaccagcgagcagattgtcgagcggttcgggttctccgagatcatg

SEQ ID NO: 25 - DNA, 354 bp, Mycobacterium tuberculosis H37Rv
cgacgacatcgaccacttcggcaaccgccgcctgcgtacggtcggcgagctgatccaaaaccagatccgggtcggcatgtcgcggatggagcgggtggtccgggagcggatgaccacccaggacgtggaggcgatcacaccgcagacgttgatcaacatccggccggtggtcgccgcgatcaaggagttcttcggcaccagccagctgagccaattcatggaccagaacaacccgctgtcggggttgacccacaagcgccgactgtcggcgctggggcccggcggtctgtcacgtgagcgtgccgggctggaggtccgcgacgtgcacccgtcgcactacggccggatgtgccc

SEQ ID NO: 26 - DNA, 542 bp, Mycobacterium tuberculosis H37Rv
acctcgatccacatcgaggagcatgagatcgatgctcgcgacaccaagctgggtgcggaggagatcacccgcgacatcccgaacatctccgacgaggtgctcgccgacctggatgagcggggcatcgtgcgcatcggtgccgaggttcgcgacggggacatcctggtcggcaaggtcaccccgaagggtgagaccgagctgacgccggaggagcggctgctgcgtgccatcttcggtgagaaggcccgcgaggtgcgcgacacttcgctgaaggtgccgcacggcgaatccggcaaggtgatcggcattcgggtgttttcccgcgaggacgaggacgagttgccggccggtgtcaacgagctggtgcgtgtgtatgtggctcagaaacgcaagatctccgacggtgacaagctggccggccggcacggcaacaagggcgtgatcggcaagatcctgccggttgaggacatgccgttccttgccgacggcaccccggtggacattattttgaacacccacggcgtgccgcgacggatgaacat

SEQ ID NO: 27 - DNA, 335 bp, Mycobacterium tuberculosis H37Rv
ggctacatgtacatcatgaagctgcaccacctggtggacgacaagatccacgcccgctccaccgggccgtactcgatgatcacccagcagccgctgggcggtaaggcgcagttcggtggccagcggttcggggagatggagtgctgggccatgcaggcctacggtgctgcctacaccctgcaggagctgttgaccatcaagtccgatgacaccgtcggccgcgtcaaggtgtacgaggcgatcgtcaagggtgagaacatcccggagccgggcatccccgagtcgttcaaggtgctgctcaaagaactgcagtcgctgtgcctcaacgtcgaggt

SEQ ID NO: 28 - DNA, 328 bp, Mycobacterium tuberculosis H37Rv
acgtgctcgacgtcaacttcttcgatgaactccgcatcggtcttgctaccgcggaggacatcaggcaatggtcctatggcgaggtcaaaaagccggagacgatcaactaccgcacgcttaagccggagaaggacggcctgttctgcgagaagatcttcgggccgactcgcgactgggaatgctactgcggcaagtacaagcgggtgcgcttcaagggcatcatctgcgagcgctgcggcgtcgaggtgacccgcgccaaggtgcgtcgtgagcggatgggccacatcgagcttgccgcgcccgtcacccacatctggtacttcaaggg

SEQ ID NO: 29 - DNA, 812 bp, Mycobacterium tuberculosis H37Rv
gtgatcccgccggagctgcgcccgatggtgcagctcgacggcggccggttcgccacgtccgacttgaacgacctgtaccgcagggtgatcaaccgcaacaaccggctgaaaaggctgatcgatctgggtgcgccggaaatcatcgtcaacaacgagaagcggatgctgcaggaatccgtggacgcgctgttcgacaatggccgccgcggccggcccgtcaccgggccgggcaaccgtccgctcaagtcgctttccgatctgctcaagggcaagcagggccggttccggcagaacctgctcggcaagcgtgtcgactactcgggccggtcggtcatcgtggtcggcccgcagctcaagctgcaccagtgcggtctgcccaagctgatggcgctggagctgttcaagccgttcgtgatgaagcggctggtggacctcaaccatgcgcagaacatcaagagcgccaagcgcatggtggagcgccagcgcccccaagtgtgggatgtgctcgaagaggtcatcgccgagcacccggtgttgctgaaccgcgcacccaccctgcaccggttgggtatccaggccttcgagccaatgctggtggaaggcaaggccattcagctgcacccgttggtgtgtgaggcgttcaatgccgacttcgacggtgaccagatggccgtgcacctgcctttgagcgccgaagcgcaggccgaggctcgcattttgatgttgtcctccaacaacatcctgtcgccggcatctgggcgtccgttggccatgccgcggctggacatggtgaccgggctgtactacctgac

SEQ ID NO: 30 - DNA, 409 bp, Mycobacterium tuberculosis H37Rv; identical to SEQ ID NO: 8

SEQ ID NO: 31 - DNA, 387 bp, Mycobacterium tuberculosis H37Rv
agtccaggaggacacaaaagtggcgaaggcgaagttccagcggaccaagccccacgtcaacatcgggaccatcggtcacgttgaccacggcaagaccaccctgaccgcggctatcaccaaggtcctgcacgacaaattccccgatctgaacgagacgaaggcattcgaccagatcgacaacgcccccgaggagcgtcagcgcggtatcaccatcaacatcgcgcacgtggagtaccagaccgacaagcggcactacgcacacgtcgacgcccctggccacgccgactacatcaagaacatgatcaccggcgccgcgcagatggacggtgcgatcctggtggtcgccgccaccgacggcccgatgccccagacccgcgagcacgttct

SEQ ID NO: 32 - DNA, 419 bp, Mycobacterium tuberculosis H37Rv
agaccaccgtcaccggtgtggagatgttccgcaagctgctcgaccagggccaggcgggcgacaacgttggtttgctgctgcggggcgtcaagcgcgaggacgtcgagcgtggccaggttgtcaccaagcccggcaccaccacgccgcacaccgagttcgaaggccaggtctacatcctgtccaaggacgagggcggccggcacacgccgttcttcaacaactaccgtccgcagttctacttccgcaccaccgacgtgaccggtgtggtgacactgccggagggcaccgagatggtgatgcccggtgacaacaccaacatctcggtgaagttgatccagcccgtcgccatggacgaaggtctgcgtttcgcgatccgcgagggtggccgcaccgtgggcgccggccgggtcaccaagatc

SEQ ID NO: 33 - DNA, 296 bp, Mycobacterium tuberculosis H37Rv; identical to SEQ ID NO: 10

SEQ ID NO: 34 - DNA, 343 bp, Mycobacterium tuberculosis H37Rv; identical to SEQ ID NO: 11

SEQ ID NO: 35 - DNA, 1449 bp, Mycobacterium tuberculosis H37Rv; identical to SEQ ID NO: 12

SEQ ID NO: 36 - DNA, 500 bp, Mycobacterium tuberculosis H37Rv
tggacccgaagcggagtgatctacccatggccagggtgaagcgcgggtaagaccgcgtggaggcccgaacccacttaggttgaagactgaggggatgagctgtgggtaggggtgaaaggccaatcaaactccgtgatagctggttctccccgaaatgcatttaggtgcagcgttgcgtggttcaccgcggaggtagagctactggatggccgatgggccctactaggttactgacgtcagccaaactccgaatgccgtggtgtaaagcgtggcagtgagacggcgggggataagctccgtacgtcgaaagggaaacagcccagatcgccggctaaggcccccaagcgtgtgctaagtgggaaaggatgtgcagtcgcaaagacaaccaggaggttggcttagaagcagccacccttgaaagagtgcgtaatagctcactggtcaagtgattgtgcgccgataatgtagcggggctcaagcacaccgccgaagccgcggca

SEQ ID NO: 37 - DNA, 1001 bp, Mycobacterium tuberculosis H37Rv
agcgactgtttactaaaaacacaggtccgtgcgaagtcgcaagacgatgtatacggactgacgcctgcccggtgctggaaggttaagaggacccgttaacccgcaagggtgaagcggagaatttaagccccagtaaacggcggtggtaactataaccatcctaaggtagcgaaattccttgtcgggtaagttccgacctgcacgaatggcgtaacgacttctcaactgtctcaaccatagactcggcgaaattgcactacgagtaaagatgctcgttacgcgcggcaggacgaaaagaccccgggaccttcactacaacttggtattgatgttcggtacggtttgtgtaggataggtgggagactgtgaaacctcgacgccagttggggcggagtcgttgttgaaataccactctgatcgtattgggcatctaacctcgaaccctgaatcgggtttagggacagtgcctggcgggtagtttaactggggcggttgcctcctaaaatgtaacggaggcgcccaaaggttccctcaacctggacggcaatcaggtggcgagtgtaaatgcacaagggagcttgactgcgagacttacaagtcaagcagggacgaaagtcgggattagtgatccggcacccccgagtggaaggggtgtcgctcaacggataaaaggtaccccggggataacaggctgatcttccccaagagtccatatcgacgggatggtttggcacctcgatgtcggctcgtcgcatcctggggctggagcaggtcccaagggttgggctgttcgcccattaaagcggcacgcgagctgggtttagaacgtcgtgagacagttcggtctctatccgccgcgcgcgtcagaaacttgaggaaacctgtccctagtacgagaggaccgggacggacgaacctctggtgcaccagttgtcccgccaggggcaccgctggatagccacgttcggtcaggataaccgctgaaagcatctaagcgggaa

SEQ ID NO: 38 - DNA, 1130 bp, Mycobacterium tuberculosis H37Rv; identical to SEQ ID NO: 14

SEQ ID NO: 39 - DNA, 416 bp, Mycobacterium tuberculosis H37Rv; identical to SEQ ID NO: 15

SEQ ID NO: 40 - DNA, 346 bp, Mycobacterium tuberculosis H37Rv
cgcagccggatgtagtacagcgtcttgatccccttgcgccaggcgtaaatctgcgccttgttcacgtcgcgggtggtggcggtgtctttgaagaacaacgtcagcgaaagcccttgatccacatgctgggtggccgccgcgtaggtgtcgatgatcttctcgtaaccgatctcgtaggcgtcttcgtagtactccaggttgtcgttggtcatatacggcgccgggtagtagacccgcccgatcttgccttccttgcggatctcgaccttcgacacgatcgggtgaatcgacgacgtcgaatggttgatgtaggaaatcgacccggtcggcggcaccgcctgcaggt

SEQ ID NO: 41 - DNA, 565 bp, Mycobacterium tuberculosis H37Rv
gtgtcttcgaacatgatataggggtagccggactcgaactgcagctcggccagcgtctggaagaactcccgtgccttgatcttggtcttgcggatgcgcgcgtcatcgaccatttcgtagtacttctcggtgaccgagatgtcagcgaacggcacaccgtagacccgctcgacatcgtagggcgagaacaggtacatgtcatcgttgcgcttggccaactcgaaggtgatgtcggggatcaccacccccagactcagcgtcttgatccggatcttctcgtcggcgttctcacgcttggtgtccaggaatcggtagatgtcggggtgatgggcgtgcaggtacaccgcgccggcaccttgacgagcgcccagctggttggcgtaggagaacgcatcctccagcaacttcatgatggggatgacgcccgaggactggttctcgatgttcttgatcggcgcgccgtgctcgcgaatgttggtcagcagcaacgccactcccccgccacgcttggatagctgcagcgcggagttgatcgaccgtccgatcgactccatgttgtcttcga

SEQ ID NO: 42 - DNA, 601 bp, Mycobacterium tuberculosis H37Rv
cttagagatgtcggaggtgcccagattggacgtaaagatcagcacggtgttcttgaagtccaccgtgcggccctgcccgtcggtgagccggccatcctcgagcacctgcagcaggctgttgtagatctcctgatgcgccttctcgatctcgtcgaacagcaccaccgagaacggcttgcgccgcaccttctcggtgagttggccgccctcctcgtagccgacgtatccgggcggcgcgccgaatagccgcgacgcggtgaaccggtcgtggaattcacccatgtcaatctgaataagcgcgtcgtcgtcaccgaacaagaagttggccagcgccttggacagttcggtcttaccgacaccggacgggccggcgaagatgaacgagcccgacgggcgcttggggtctttcagcccggcccgggtacgccggatggccttggaaacggccttgacggcgtcctcttgcccgatgatccgcttgtgcagctcttcttccatccgcaacagccgggtggtctcggcctcggtgagcttgaacaccgggataccggtccagttgcccagcacctcggcgatctgctcgtcgtcgacctccgcgacc

SEQ ID NO: 43 - DNA, 616 bp, Mycobacterium tuberculosis H37Rv
catcggtgatcgacacccggtggtgcgcctcgtaccggtcccgcaggcccttgaggatctcgatggtgtgctccaccgtcggctcacccacctgcaccggctggaagcggcgctccagcgcggcgtccttctcgatgtacttgcggtattcgtcgagcgtggtggcgccgatcgtttgcagttcaccgcgagcgagcttcggtttcaggatcgaggcggcgtcgatcgcgccctcggcggctccagcaccgaccaaggtgtgcagctcgtcgataaacaggatgatgtcaccgcgggtgttgatctccttgagcaccttcttgaggcgttcctcgaagtcaccgcggtagcggctacccgccaccagcgatcccagatccagcgtgtagagctgcttgtccttgagcgtctcgggcacctcgccgtgcacgatggcctgcgccagtccttcgacgaccgcggtcttgccgacgccgggctcgccgatcagcaccgggttgttcttggtgcgccgagagagcacctgcatgacccgctcgatttccttctcgcggccgatgaccgggtccagtttgccttccatcgccgccgccgtgaggttgcggccgaactggtc

SEQ ID NO: 44 - DNA, 338 bp, Mycobacterium tuberculosis H37Rv; identical to SEQ ID NO: 18

SEQ ID NO: 45 - DNA, 4900 bp, Mycobacterium tuberculosis H37Rv
cagacggtgttcatgggtgacttcccgatgatgaccgagaagggcacgttcatcatcaacgggaccgagcgtgtggtggtcagccagctggtgcggtcgcccggggtgtacttcgacgagaccattgacaagtccaccgacaagacgctgcacagcgtcaaggtgatcccgagccgcggcgcgtggctcgagtttgacgtcgacaagcgcgacaccgtcggcgtgcgcatcgaccgcaaacgccggcaaccggtcaccgtgctgctcaaggcgctgggctggaccagcgagcagattgtcgagcggttcgggttctccgagatcatgcgatcgacgctggagaaggacaacaccgtcggcaccgacgaggcgctgttggacatctaccgcaagctgcgtccgggcgagcccccgaccaaagagtcagcgcagacgctgttggaaaacttgttcttcaaggagaagcgctacgacctggcccgcgtcggtcgctataaggtcaacaagaagctcgggctgcatgtcggcgagcccatcacgtcgtcgacgctgaccgaagaagacgtcgtggccaccatcgaatatctggtccgcttgcacgagggtcagaccacgatgaccgttccgggcggcgtcgaggtgccggtggaaaccgacgacatcgaccacttcggcaaccgccgcctgcgtacggtcggcgagctgatccaaaaccagatccgggtcggcatgtcgcggatggagcgggtggtccgggagcggatgaccacccaggacgtggaggcgatcacaccgcagacgttgatcaacatccggccggtggtcgccgcgatcaaggagttcttcggcaccagccagctgagccaattcatggaccagaacaacccgctgtcggggttgacccacaagcgccgactgtcggcgctggggcccggcggtctgtcacgtgagcgtgccgggctggaggtccgcgacgtgcacccgtcgcactacggccggatgtgcccgatcgaaacccctgaggggcccaacatcggtctgatcggctcgctgtcggtgtacgcgcgggtcaacccgttcgggttcatcgaaacgccgtaccgcaaggtggtcgacggcgtggttagcgacgagatcgtgtacctgaccgccgacgaggaggaccgccacgtggtggcacaggccaattcgccgatcgatgcggacggtcgcttcgtcgagccgcgcgtgctggtccgccgcaaggcgggcgaggtggagtacgtgccctcgtctgaggtggactacatggacgtctcgccccgccagatggtgtcggtggccaccgcgatgattcccttcctggagcacgacgacgccaaccgtgccctcatgggggcaaacatgcagcgccaggcggtgccgctggtccgtagcgaggccccgctggtgggcaccgggatggagctgcgcgcggcgatcgacgccggcgacgtcgtcgtcgccgaagaaagcggcgtcatcgaggaggtgtcggccgactacatcactgtgatgcacgacaacggcacccggcgtacctaccggatgcgcaagtttgcccggtccaaccacggcacttgcgccaaccagtgccccatcgtggacgcgggcgaccgagtcgaggccggtcaggtgatcgccgacggtccctgtactgacgacggcgagatggcgctgggcaagaacctgctggtggccatcatgccgtgggagggccacaactacgaggacgcgatcatcctgtccaaccgcctggtcgaagaggacgtgctcacctcgatccacatcgaggagcatgagatcgatgctcgcgacaccaagctgggtgcggaggagatcacccgcgacatcccgaacatctccgacgaggtgctcgccgacctggatgagcggggcatcgtgcgcatcggtgccgaggttcgcgacggggacatcctggtcggcaaggtcaccccgaagggtgagaccgagctgacgccggaggagcggctgctgcgtgccatcttcggtgagaaggcccgcgaggtgcgcgacacttcgctgaaggtgccgcacggcgaatccggcaaggtgatcggcattcgggtgttttcccgcgaggacgaggacgagttgccggccggtgtcaacgagctggtgcgtgtgtatgtggctcagaaacgcaagatctccgacggtgacaagctggccggccggcacggcaacaagggcgtgatcggcaagatcctgccggttgaggacatgccgttccttgccgacggcaccccggtggacattattttgaacacccacggcgtgccgcgacggatgaacatcggccagattttggagacccacctgggttggtgtgcccacagcggctggaaggtcgacgccgccaagggggttccggactgggccgccaggctgcccgacgaactgctcgaggcgcagccgaacgccattgtgtcgacgccggtgttcgacggcgcccaggaggccgagctgcagggcctgttgtcgtgcacgctgcccaaccgcgacggtgacgtgctggtcgacgccgacggcaaggccatgctcttcgacgggcgcagcggcgagccgttcccgtacccggtcacggttggctacatgtacatcatgaagctgcaccacctggtggacgacaagatccacgcccgctccaccgggccgtactcgatgatcacccagcagccgctgggcggtaaggcgcagttcggtggccagcggttcggggagatggagtgctgggccatgcaggcctacggtgctgcctacaccctgcaggagctgttgaccatcaagtccgatgacaccgtcggccgcgtcaaggtgtacgaggcgatcgtcaagggtgagaacatcccggagccgggcatccccgagtcgttcaaggtgctgctcaaagaactgcagtcgctgtgcctcaacgtcgaggtgctatcgagtgacggtgcggcgatcgaactgcgcgaaggtgaggacgaggacctggagcgggccgcggccaacctgggaatcaatctgtcccgcaacgaatccgcaagtgtcgaggatcttgcgtaaagctgtcgcaaaattactaaacccgttaggggaaagggagttacgtgctcgacgtcaacttcttcgatgaactccgcatcggtcttgctaccgcggaggacatcaggcaatggtcctatggcgaggtcaaaaagccggagacgatcaactaccgcacgcttaagccggagaaggacggcctgttctgcgagaagatcttcgggccgactcgcgactgggaatgctactgcggcaagtacaagcgggtgcgcttcaagggcatcatctgcgagcgctgcggcgtcgaggtgacccgcgccaaggtgcgtcgtgagcggatgggccacatcgagcttgccgcgcccgtcacccacatctggtacttcaagggtgtgccctcgcggctggggtatctgctggacctggccccgaaggacctggagaagatcatctacttcgctgcctacgtgatcacctcggtcgacgaggagatgcgccacaatgagctctccacgctcgaggccgaaatggcggtggagcgcaaggccgtcgaagaccagcgcgacggcgaactagaggcccgggcgcaaaagctggaggccgacctggccgagctggaggccgagggcgccaaggccgatgcgcggcgcaaggttcgcgacggcggcgagcgcgagatgcgccagatccgtgaccgcgcgcagcgtgagctggaccggttggaggacatctggagcactttcaccaagctggcgcccaagcagctgatcgtcgacgaaaacctctaccgcgaactcgtcgaccgctacggcgagtacttcaccggtgccatgggcgcggagtcgatccagaagctgatcgagaacttcgacatcgacgccgaagccgagtcgctgcgggatgtcatccgaaacggcaaggggcagaagaagcttcgcgccctcaagcggctgaaggtggttgcggcgttccaacagtcgggcaactcgccgatgggcatggtgctcgacgccgtcccggtgatcccgccggagctgcgcccgatggtgcagctcgacggcggccggttcgccacgtccgacttgaacgacctgtaccgcagggtgatcaaccgcaacaaccggctgaaaaggctgatcgatctgggtgcgccggaaatcatcgtcaacaacgagaagcggatgctgcaggaatccgtggacgcgctgttcgacaatggccgccgcggccggcccgtcaccgggccgggcaaccgtccgctcaagtcgctttccgatctgctcaagggcaagcagggccggttccggcagaacctgctcggcaagcgtgtcgactactcgggccggtcggtcatcgtggtcggcccgcagctcaagctgcaccagtgcggtctgcccaagctgatggcgctggagctgttcaagccgttcgtgatgaagcggctggtggacctcaaccatgcgcagaacatcaagagcgccaagcgcatggtggagcgccagcgcccccaagtgtgggatgtgctcgaagaggtcatcgccgagcacccggtgttgctgaaccgcgcacccaccctgcaccggttgggtatccaggccttcgagccaatgctggtggaaggcaaggccattcagctgcacccgttggtgtgtgaggcgttcaatgccgacttcgacggtgaccagatggccgtgcacctgcctttgagcgccgaagcgcaggccgaggctcgcattttgatgttgtcctccaacaacatcctgtcgccggcatctgggcgtccgttggccatgccgcggctggacatggtgaccgggctgtactacctgac

SEQ ID NO: 46 - DNA, 4721 bp, Mycobacterium tuberculosis H37Rv
gagtggcgaacgggtgagtaacacgtgggtgatctgccctgcacttcgggataagcctgggaaactgggtctaataccggataggaccacgggatgcatgtcttgtggtggaaagcgctttagcggtgtgggatgagcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcaccatcgacgaaggtccgggttctctcggattgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcacgtaatggtggggactcgtgagagactgccggggtcaactcggaggaaggtggggatgacgtcaagtcatcatgccccttatgtccagggcttcacacatgctacaatggccggtacaaagggctgcgatgccgcgaggttaagcgaatccttaaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaaccctcgggagggagctgtcgaaggtgggatcggcgattgggacgaagtcgtaacaaggtagccgtaccggaaggtgcggctggatcacctcctttctaaggagcaccacgaaaacgccccaactggtggggcgtaggccgtgaggggttcttgtctgtagtgggcgagagccgggtgcatgacaacaaagttggccaccaacacactgttgggtcctgaggcaacactcggacttgttccaggtgttgtcccaccgccttggtggtggggtgtggtgtttgagaactggatagtggttgcgagcatcaatggatacgctgccggctagcggtggcgtgttctttgtgcaatattctttggtttttgttgtgtttgtaagtgtctaagggcgcatggtggatgccttggcatcgagagccgatgaaggacgtgggaggctgcgatatgcctcggggagctgtcaaccgagcgtggatccgaggatttccgaatggggaaacccagcacgagtgatgtcgtgctacccgcatctgaatatatagggtgcgggagggaacgcggggaagtgaaacatctcagtacccgtaggaggagaaaacaattgtgattccgcaagtagtggcgagcgaacgcggaacaggctaaaccgcacgcatgggtaaccgggtaggggttgtgtgtgcggggttgtgggaggatatgtctcagcgctacccggctgagaggcagtcagaaagtgtcgtggttagcggaagtggcctgggatggtctgccgtagacggtgagagcccggtacgcgaaaacccggcacctgcctagtatcaattcccgagtagcagcgggcccgtggaatccgctgtgaatccgccgggaccacccggtaagcctaaatactcctcgatgaccgatagcggattagtaccgtgagggaatggtgaaaagtaccccgggaggggagtgaaagagtacctgaaaccgtgtgcctacaatccgtcagagcctccttttcctctccggaggagggtggtgatggcgtgccttttgaagaatgagcctgcgagtcagggacatgtcgcaaggttaacccgtgtggggtagccgcagcgaaagcgagtctgaatagggcgacccacacgcgcatacgcgcgtgtgaatagtggcgtgttctggacccgaagcggagtgatctacccatggccagggtgaagcgcgggtaagaccgcgtggaggcccgaacccacttaggttgaagactgaggggatgagctgtgggtaggggtgaaaggccaatcaaactccgtgatagctggttctccccgaaatgcatttaggtgcagcgttgcgtggttcaccgcggaggtagagctactggatggccgatgggccctactaggttactgacgtcagccaaactccgaatgccgtggtgtaaagcgtggcagtgagacggcgggggataagctccgtacgtcgaaagggaaacagcccagatcgccggctaaggcccccaagcgtgtgctaagtgggaaaggatgtgcagtcgcaaagacaaccaggaggttggcttagaagcagccacccttgaaagagtgcgtaatagctcactggtcaagtgattgtgcgccgataatgtagcggggctcaagcacaccgccgaagccgcggcacatccaccttgtggtgggtgtgggtaggggagcgtccctcattcagcgaagccaccgggtgaccggtggtggagggtgggggagtgagaatgcaggcatgagtagcgacaaggcaagtgagaaccttgcccgccgaaagaccaagggttcctgggccaggccagtccgcccagggtgagtcgggacctaaggcgaggccgacaggcgtagtcgatggacaacgggttgatattcccgtacccgtgtgtgggcgcccgtgacgaatcagcggtactaaccacccaaaaccggatcgatcactccccttcgggggtgtggagttctggggctgcgtgggaacttcgctggtagtagtcaagcgaaggggtgacgcaggaaggtagccgtaccagtcagtggtaacactggggcaagccggtagggagagcgataggcaaatccgtcgctcactaatcctgagaggtgacgcatagccggttgaggcgaattcggtgatcctctgctgccaagaaaagcctctagcgagcacacacacggcccgtaccccaaaccgacacaggtggtcaggtagagcataccaaggcgtacgagataactatggttaaggaactcggcaaaatgcccccgtaacttcgggagaagggggaccggaatatcgtgaacacccttgcggtgggagcgggatccggtcgcagaaaccagtgaggagcgactgtttactaaaaacacaggtccgtgcgaagtcgcaagacgatgtatacggactgacgcctgcccggtgctggaaggttaagaggacccgttaacccgcaagggtgaagcggagaatttaagccccagtaaacggcggtggtaactataaccatcctaaggtagcgaaattccttgtcgggtaagttccgacctgcacgaatggcgtaacgacttctcaactgtctcaaccatagactcggcgaaattgcactacgagtaaagatgctcgttacgcgcggcaggacgaaaagaccccgggaccttcactacaacttggtattgatgttcggtacggtttgtgtaggataggtgggagactgtgaaacctcgacgccagttggggcggagtcgttgttgaaataccactctgatcgtattgggcatctaacctcgaaccctgaatcgggtttagggacagtgcctggcgggtagtttaactggggcggttgcctcctaaaatgtaacggaggcgcccaaaggttccctcaacctggacggcaatcaggtggcgagtgtaaatgcacaagggagcttgactgcgagacttacaagtcaagcagggacgaaagtcgggattagtgatccggcacccccgagtggaaggggtgtcgctcaacggataaaaggtaccccggggataacaggctgatcttccccaagagtccatatcgacgggatggtttggcacctcgatgtcggctcgtcgcatcctggggctggagcaggtcccaagggttgggctgttcgcccattaaagcggcacgcgagctgggtttagaacgtcgtgagacagttcggtctctatccgccgcgcgcgtcagaaacttgaggaaacctgtccctagtacgagaggaccgggacggacgaacctctggtgcaccagttgtcccgccaggggcaccgctggatagccacgttcggtcaggataaccgctgaaagcatctaagcgggaa